(19) United States
(12) Patent Application Publication
(10) Pub. No.: US 2020/0085828 A1
(43) Pub. Date: Mar. 19, 2020

(54) PHOSPHODIESTERASE INHIBITORS AND METHODS OF MICROBIAL TREATMENT

(71) Applicant: Mavupharma, Inc., Kirkland, WA (US)

(72) Inventors: William Michael GALLATIN, Kirkland, WA (US); Gregory N. DIETSCH, Kirkland, WA (US)

(21) Appl. No.: 16/470,529

(22) PCT Filed: Dec. 21, 2017
(86) PCT No.: PCT/US2017/068044

§ 371 Date: Jun. 17, 2019

Related U.S. Application Data
(60) Provisional application No. 62/438,344, filed on Dec. 22, 2016.

(51) Int. Cl. A61K 31/519; C07K 14/47; C12N 9/80

(52) U.S. Cl. CPC A61K 31/519; A61K 45/06; C12N 9/80; C07K 14/473

(57) ABSTRACT

Disclosed herein are methods and compounds for treating augmenting and enhancing the production of type I IFNs in vivo. In some embodiments, also disclosed herein include methods of activating and enhancing the cGAS-STING response and use of an inhibitor of a phosphodiesterase for the treatment of a microbial infection.

### CROSS-REFERENCE

This patent application claims the benefit of U.S. Provisional Patent Application No. 62/438,344, filed Dec. 22, 2016, which is incorporated herein by reference in its entirety.

### BACKGROUND OF THE DISCLOSURE

Microorganisms that are capable of causing disease are termed pathogens. Pathogenic microorganisms include bacteria, virus, fungi, protozoa, and helminths. In some instances, antimicrobials such as broad spectrum fluoroquinolones and oxazolidinones fight infection by inhibiting microbial reproduction within a host. In other instances, antimicrobials enhance or strengthen a host's immune response to the pathogenic infection.

### SUMMARY OF THE DISCLOSURE

Disclosed herein, in certain embodiments, are methods of augmenting and/or enhancing the production of type I IFNs in vivo. In some embodiments, disclosed herein comprise methods of treating a pathogenic infection by administering an inhibitor of 2′3′-cGAMP degradation polypeptide (e.g., an inhibitor of a phosphodiesterase). In other embodiments, also described herein are designs and generation of selective inhibitors to prevent the degradation of a STING activating substrate and pharmaceutical compositions comprising the selective inhibitor.

Disclosed herein, in certain embodiments, is a method of treating a subject in need thereof, comprising: administering to the subject an inhibitor of a 2′3′-cGAMP degradation polypeptide, wherein the inhibitor prevents hydrolysis of 2′3′-cGAMP and wherein the subject has an infection. In some embodiments, the 2′3′-cGAMP degradation polypeptide is a phosphodiesterase (PDE). In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the inhibitor is a small molecule. In some embodiments, the inhibitor is a PDE inhibitor. In some embodiments, the inhibitor is a ENPP-1 inhibitor. In some embodiments, the inhibitor is a reversible inhibitor. In some embodiments, the inhibitor is a competitive inhibitor. In some embodiments, the inhibitor is an allosteric inhibitor. In some embodiments, the inhibitor is an irreversible inhibitor. In some embodiments, the inhibitor is a mixed inhibitor. In some embodiments, the inhibitor binds to the catalytic domain of ENPP-1. In some embodiments, the inhibitor binds to the nuclease-like domain of ENPP-1. In some embodiments, the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the infection is a viral infection. In some embodiments, the viral infection is due to a DNA virus. In some embodiments, the viral infection is due to a retrovirus. In some embodiments, the viral infection is due to herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In some embodiments, the infection is a bacterial infection. In some embodiments, the bacterial infection is due to a Gram-negative bacterium. In some embodiments, the bacterial infection is due to a Gram-positive bacterium. In some embodiments, the bacterial infection is due to Listeria monocytogenes, Mycobacterium tuberculosis, Francisella novicida, Legionella pneumophila, Chlamydia trachomatis, Streptococcus pneumoniae, or Neisseria gonorrhoeae. In some embodiments, the inhibitor is administered continuously for 1, 2, 3, 4, 5, 6, 7, 8, 9, 10, 14, 15, 28, 30 or more days. In some embodiments, the inhibitor is administered at predetermined time intervals for 1, 2, 3, 4, 5, 6, 7, 8, 9, 10, 14, 15, 28, 30 or more days. In some embodiments, the inhibitor is administered intermittently for 1, 2, 3, 4, 5, 6, 7, 8, 9, 10, 14, 15, 28, 30 or more days. In some embodiments, the inhibitor is administered to the subject at a therapeutically effective amount. In some embodiments, the therapeutically effective amount is administered in 1 dose, 2 doses, 3 doses, 4 doses, 5 doses, 6 doses or more. In some embodiments, the therapeutically effective amount of the inhibitor selectively inhibits hydrolysis of 2′3′-cGAMP. In some embodiments, the inhibitor has a reduced inhibition function of ATP hydrolysis of the 2′3′-cGAMP degradation polypeptide. In some embodiments, the inhibitor reduces ATP hydrolysis in the 2′3′-cGAMP degradation polypeptide by less than 50%, less than 40%, less than 30%, less than 20%, less than 10%, less than 5%, or to less than 1% relative to the ATP hydrolysis of a 2′3′-cGAMP degradation polypeptide in the absence of the inhibitor. In some embodiments, the inhibitor does not inhibit ATP hydrolysis in the 2′3′-cGAMP degradation polypeptide. In some embodiments, the method further comprises administering an additional therapeutic agent. In some embodiments, the additional therapeutic agent is an antimicrobial agent. In some embodiments, the inhibitor and the additional therapeutic agent is administered simultaneously. In some embodiments, the inhibitor and the additional therapeutic agent is administered sequentially. In some embodiments, the inhibitor is administered before administering the additional therapeutic agent. In some embodiments, the inhibitor is administered after administering the additional therapeutic agent. In some embodiments, the subject is a human.

Disclosed herein, in certain embodiments, is a method of inhibiting depletion of 2′3′-cGAMP in a cell infected by a pathogen, comprising: contacting the cell infected by a pathogen and expressing a 2′3′-cGAMP degradation polypeptide with an inhibitor to generate a 2′3′-cGAMP degradation polypeptide-inhibitor adduct, thereby inhibiting the 2′3′-cGAMP degradation polypeptide from degrading 2′3′-cGAMP to prevent the depletion of 2′3′-cGAMP in the cell. In some embodiments, the 2′3′-cGAMP degradation polypeptide is a phosphodiesterase (PDE). In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the inhibitor is a small molecule. In some embodiments, the inhibitor is a PDE inhibitor. In some embodiments, the inhibitor is a ENPP-1 inhibitor. In some embodiments, the inhibitor is a reversible inhibitor. In some embodiments, the inhibitor is a competitive inhibitor. In some embodiments, the inhibitor is an allosteric inhibitor. In some embodiments, the inhibitor is an irreversible inhibitor. In some embodiments, the inhibitor is a mixed inhibitor. In some embodiments, the inhibitor binds to the catalytic domain of ENPP-1. In some embodiments, the inhibitor binds to the nuclease-like domain of ENPP-1. In some embodiments, the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the pathogen is a virus. In some embodiments, the virus is a DNA virus. In some embodiments, the virus is a retrovirus. In some embodiments, the virus is herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In some embodiments, the pathogen is a bacterium. In some embodiments, the bacterium is a Gram-negative bacterium. In some embodiments, the bacterium is a Gram-positive bacterium. In some embodiments, the bacterium is Listeria monocytogenes, Mycobacterium tuberculosis, Francisella novicida, Legionella pneumophila, Chlamydia trachomatis, Streptococcus pneumoniae, or Neisseria gonorrhoeae. In some embodiments, the cell is further characterized by an elevated population of cytosolic DNA. In some embodiments, the method is an in vivo method.

Disclosed herein, in certain embodiments, is a method of enhancing type I interferon (IFN) production in a subject having an infection due to a pathogen, comprising: administering to the subject having an infection due to a pathogen a pharmaceutical composition comprising: (i) an inhibitor of a 2′3′-cGAMP degradation polypeptide to block the hydrolysis of 2′3′-cGAMP; and (ii) a pharmaceutically acceptable excipient; wherein the presence of 2′3′-cGAMP activates the STING pathway, thereby enhancing the production of type I interferons. In some embodiments, the 2′3′-cGAMP degradation polypeptide is a phosphodiesterase (PDE). In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the inhibitor is a small molecule. In some embodiments, the inhibitor is a PDE inhibitor. In some embodiments, the inhibitor is a ENPP-1 inhibitor. In some embodiments, the inhibitor is a reversible inhibitor. In some embodiments, the inhibitor is a competitive inhibitor. In some embodiments, the inhibitor is an allosteric inhibitor. In some embodiments, the inhibitor is an irreversible inhibitor. In some embodiments, the inhibitor is a mixed inhibitor. In some embodiments, the inhibitor binds to the catalytic domain of ENPP-1. In some embodiments, the inhibitor binds to the nuclease-like domain of ENPP-1. In some embodiments, the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the pathogen is a virus. In some embodiments, the virus is a DNA virus. In some embodiments, the virus is a retrovirus. In some embodiments, the virus is herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In some embodiments, the pathogen is a bacterium. In some embodiments, the bacterium is a Gram-negative bacterium. In some embodiments, the bacterium is a Gram-positive bacterium. In some embodiments, the bacterium is Listeria monocytogenes, Mycobacterium tuberculosis, Francisella novicida, Legionella pneumophila, Chlamydia trachomatis, Streptococcus pneumoniae, or Neisseria gonorrhoeae. In some embodiments, the inhibitor is administered continuously for 1, 2, 3, 4, 5, 6, 7, 8, 9, 10, 14, 15, 28, 30 or more days. In some embodiments, the inhibitor is administered at predetermined time intervals for 1, 2, 3, 4, 5, 6, 7, 8, 9, 10, 14, 15, 28, 30 or more days. In some embodiments, the inhibitor is administered intermittently for 1, 2, 3, 4, 5, 6, 7, 8, 9, 10, 14, 15, 28, 30 or more days. In some embodiments, the inhibitor is administered to the subject at a therapeutically effective amount. In some embodiments, the therapeutically effective amount is administered in 1 dose, 2 doses, 3 doses, 4 doses, 5 doses, 6 doses or more. In some embodiments, the therapeutically effective amount of the inhibitor selectively inhibits hydrolysis of 2′3′-cGAMP but not ATP hydrolysis in the 2′3′-cGAMP degradation polypeptide. In some embodiments, the method further comprises administering an additional therapeutic agent. In some embodiments, the additional therapeutic agent is an antimicrobial agent. In some embodiments, the inhibitor and the additional therapeutic agent is administered simultaneously. In some embodiments, the inhibitor and the additional therapeutic agent is administered sequentially. In some embodiments, the inhibitor is administered before administering the additional therapeutic agent. In some embodiments, the inhibitor is administered after administering the additional therapeutic agent. In some embodiments, the subject is a human.

Disclosed herein, in certain embodiments, is a method of stabilizing a stimulator of interferon genes (STING) protein dimer in a cell infected by a pathogen, comprising: (a) contacting the cell infected by a pathogen and characterized with an elevated population of cytosolic DNA with an inhibitor of a 2′3′-cGAMP degradation polypeptide to inhibit hydrolysis of 2′3′-cGAMP; and (b) interacting 2′3′-cGAMP to a STING protein dimer to generate a 2′3′-cGAMP-STING complex, thereby stabilizing the STING protein dimer. In some embodiments, the 2′3′-cGAMP degradation polypeptide is a phosphodiesterase (PDE). In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the inhibitor is a small molecule. In some embodiments, the inhibitor is a PDE inhibitor. In some embodiments, the inhibitor is a ENPP-1 inhibitor. In some embodiments, the inhibitor is a reversible inhibitor. In some embodiments, the inhibitor is a competitive inhibitor. In some embodiments, the inhibitor is an allosteric inhibitor. In some embodiments, the inhibitor is an irreversible inhibitor. In some embodiments, the inhibitor is a mixed inhibitor. In some embodiments, the inhibitor binds to the catalytic domain of ENPP-1. In some embodiments, the inhibitor binds to the nuclease-like domain of ENPP-1. In some embodiments, the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the pathogen is a virus. In some embodiments, the virus is a DNA virus. In some embodiments, the virus is a retrovirus. In some embodiments, the virus is herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In some embodiments, the pathogen is a bacterium. In some embodiments, the bacterium is a Gram-negative bacterium. In some embodiments, the bacterium is a Gram-positive bacterium. In some embodiments, the bacterium is Listeria monocytogenes, Mycobacterium tuberculosis, Francisella novicida, Legionella pneumophila, Chlamydia trachomatis, Streptococcus pneumoniae, or Neisseria gonorrhoeae. In some embodiments, the method is an in vivo method.

Disclosed herein, in certain embodiments, is a method of selectively inhibits a phosphodiesterase (PDE), comprising: contacting a cell characterized with an elevated population of cytosolic DNA with a PDE inhibitor to inhibit hydrolysis of 2′3-cGAMP, wherein the PDE inhibitor has a reduced function of ATP hydrolysis of the PDE, and wherein the elevated population of cytosolic DNA is generated by a virus. In some embodiments, also disclosed herein is a method of selectively inhibits a phosphodiesterase (PDE), comprising: contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a virus. In some embodiments, additional disclosed herein is a method of selectively inhibits a phosphodiesterase (PDE), comprising: contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a virus. In some embodiments, the cytosolic DNA are viral DNA. In some embodiments, the elevated population of cytosolic DNA is due to infection of a virus to the cell. In some embodiments, the elevated population of cytosolic DNA is due to delivery of viral DNA through a virus-like particle (VLP). In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the inhibitor is a small molecule. In some embodiments, the inhibitor is a PDE inhibitor. In some embodiments, the inhibitor is a ENPP-1 inhibitor. In some embodiments, the inhibitor is a reversible inhibitor. In some embodiments, the inhibitor is a competitive inhibitor. In some embodiments, the inhibitor is an allosteric inhibitor. In some embodiments, the inhibitor is an irreversible inhibitor. In some embodiments, the inhibitor is a mixed inhibitor. In some embodiments, the inhibitor binds to the catalytic domain of ENPP-1. In some embodiments, the inhibitor binds to the nuclease-like domain of ENPP-1. In some embodiments, the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the reduced inhibition function of ATP hydrolysis is relative to the ATP hydrolysis of a PDE in the absence of the PDE inhibitor. In some embodiments, the PDE inhibitor reduces ATP hydrolysis in the PDE by less than 50%, less than 40%, less than 30%, less than 20%, less than 10%, less than 5%, or to less than 1% relative to the ATP hydrolysis of a PDE in the absence of the PDE inhibitor. In some embodiments, the PDE inhibitor does not inhibit ATP hydrolysis of the PDE. In some embodiments, the virus is a DNA virus. In some embodiments, the virus is a retrovirus. In some embodiments, the virus is herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In some embodiments, the method is an in vivo method.

Disclosed herein, in certain embodiments, is a method of selectively inhibits a phosphodiesterase (PDE), comprising: contacting a cell characterized with an elevated population of cytosolic DNA with a PDE inhibitor to inhibit hydrolysis of 2′3-cGAMP, wherein the PDE inhibitor has a reduced inhibition function ATP hydrolysis of the PDE, and wherein the elevated population of cytosolic DNA is generated by a recombinant DNA vaccine. In some embodiments, also disclosed herein is a method of selectively inhibits a phosphodiesterase (PDE), comprising: contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a recombinant DNA vaccine. In some embodiments, additionally disclosed herein is a method of selectively inhibits a phosphodiesterase (PDE), comprising: contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a recombinant DNA vaccine. In some embodiments, the recombinant DNA vaccine comprises a DNA vector that encodes a viral antigen. In some embodiments, the viral antigen is derived from a DNA virus. In some embodiments, the viral antigen is derived from a retrovirus. In some embodiments, the viral antigen is derived from herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In some embodiments, the recombinant DNA vaccine comprises a DNA vector that encodes a bacterial antigen. In some embodiments, the bacterial antigen is derived from a Gram-negative bacterium. In some embodiments, the bacterial antigen is derived from a Gram-positive bacterium. In some embodiments, the bacterial antigen is derived from Listeria monocytogenes, Mycobacterium tuberculosis, Francisella novicida, Legionella pneumophila, Chlamydia trachomatis, Streptococcus pneumoniae, or Neisseria gonorrhoeae. In some embodiments, the method further comprises contacting the cell with the recombinant DNA vaccine. In some embodiments, the PDE comprises an ectonucleotide pyrophosphatase/phosphodiesterase (ENPP) protein. In some embodiments, the ENPP protein comprises ectonucleotide pyrophosphatase/phosphodiesterase family member 1 (ENPP-1). In some embodiments, the inhibitor is a small molecule. In some embodiments, the inhibitor is a PDE inhibitor. In some embodiments, the inhibitor is a ENPP-1 inhibitor. In some embodiments, the inhibitor is a reversible inhibitor. In some embodiments, the inhibitor is a competitive inhibitor. In some embodiments, the inhibitor is an allosteric inhibitor. In some embodiments, the inhibitor is an irreversible inhibitor. In some embodiments, the inhibitor is a mixed inhibitor. In some embodiments, the inhibitor binds to the catalytic domain of ENPP-1. In some embodiments, the inhibitor binds to the nuclease-like domain of ENPP-1. In some embodiments, the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some embodiments, the PDE inhibitor comprises 2-(3H-imidazo[4,5-b]pyridin-2-ylthio)-N-(3,4-dimethoxyphenyl)acetamide or a derivative, analog, or salt thereof. In some embodiments, the PDE inhibitor comprises 2-(6-Amino-9H-purin-8-ylthio)-N-(3,4-dimethoxyphenyl)-acetamide, or a salt thereof. In some embodiments, the PDE inhibitor comprises N-(3,4-Dimethoxyphenyl)-2-(5-methoxy-3H-imidazo[4,5-b]-pyridin-2-ylthio)acetamide or a salt thereof. In some embodiments, the PDE inhibitor comprises 2-(1-(6,7-Dimethoxyquinazolin-4-yl)piperidin-4-yl)ethyl sulfamide or a salt thereof. In some embodiments, the PDE inhibitor comprises ((1-(6,7-Dimethoxyquinazolin-4-yl)piperidin-4-yl)methyl)sulfamide or a salt thereof. In some embodiments, the PDE inhibitor comprises SK4A (SAT0037) or a derivative or salt thereof. In some embodiments, the PDE inhibitor comprises Compound 1, Compound 2, Compound 3, or a derivative, analog, or salt thereof. In some embodiments, the reduced inhibition function of ATP hydrolysis is relative to the ATP hydrolysis of a PDE in the absence of the PDE inhibitor. In some embodiments, the PDE inhibitor reduces ATP hydrolysis in the PDE by less than 50%, less than 40%, less than 30%, less than 20%, less than 10%, less than 5%, or to less than 1% relative to the ATP hydrolysis of a PDE in the absence of the PDE inhibitor. In some embodiments, the PDE inhibitor does not inhibit ATP hydrolysis of the PDE.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a cartoon representation of the cGAS-STING pathway.

FIG. 2A-FIG. 2C are exemplary bar graphs illustrating augmentation of cGAMP mediated IFNβ production in the presence of PDE inhibitor Compound 1 (FIG. 2A), Compound 2 (FIG. 2B), and Compound 3 (FIG. 2C).

### DETAILED DESCRIPTION OF THE DISCLOSURE

The innate immune system is the first line of defense to a microbial infection. The host innate immunity is activated through recognition of conserved microbial signatures termed pathogen-associated molecular patterns (PAMPs) and host damage-associated molecular patterns (DAMPs). Upon sensing of microbial PAMPs and DAMPs, signal cascades are activated to produce type I interferons and/or multiple cytokines and chemokines, culminating in the synthesis of antiviral proteins. The presence of antiviral proteins and cytokines (e.g., interferons or chemokines) subsequently promote apoptosis, inhibits cellular protein translation, and recruit immune cells to the site of infection to further initiate adaptive immune response.

Pattern recognition receptors (PRRs) are germ-line encoded receptors that recognize PAMPs and DAMPs and facilitate the rapid and efficient innate immune response. Cytosolic DNA sensor is a type of PRRs that detects the intracellular presence of pathogenic DNA. DNA-dependent activator of IFN-regulatory factors (DAI), a cytosolic DNA sensor, utilizes the cGAS-STING pathway for production of type I interferons.

Mitochondria play a role in host immune response, for example, by boosting immune cell activation and antimicrobial defense. Mitochondrial DNA (mtDNA) triggers innate immune responses when exposed during cellular stress, infection or injury. Both cytosolic and extracellular mtDNA are recognized by PRRs and trigger type I interferons and interferon-stimulated gene (ISG) expression. In some instances, cytosolic mtDNA is recognized by PRRs and triggers type I interferons and interferon-stimulated gene (ISG) expression. In some instances, mtDNA is released during apoptosis mediated by BCL-2 like protein 4 (BAX) and BCL-2 homologous antagonist/killer (BAK). In some instances, mtDNA released during apoptosis engage cGAS-STING-IRF3 signaling and trigger type I IFN responses and expression of ISGs. In some instances, mitochondrial stress liberates cytosolic mtDNA which triggers type I IFN via the cGAS-STING pathway. In some instances, the stress is pathogen-mediated. In some embodiments, infection due to a viral pathogen induces release of mitochondrial DNA (mtDNA). In some embodiments, the release of mtDNA due to a viral pathogen induces a cGAS-induced antiviral response. In some instances, the viral pathogen is a ssRNA virus. In some instances, the viral pathogen is a dengue virus. In some instances, extracellular mtDNA is recognized by PRRs and triggers type I interferons and interferon-stimulated gene (ISG) expression. Neutrophil extracellular trap (NET) formation—a process involved in bacterial clearance and sterile inflammatory diseases—results in cell death and extrusion of neutrophil DNA and/or protein complexes into the extracellular space. In some instances, extracellular mtDNA, such as mtDNA released from activated neutrophils, engage cGAS-STING pathway to trigger a type I IFN response.

In some instances, ligand for the cytosolic DNA sensor is nuclear DNA. In some instances, ligand for the cytosolic DNA sensor is mitochondrial DNA. In some instances, ligand for the cytosolic DNA sensor is cytosolic mitochondrial DNA. In some instances, ligand for the cytosolic DNA sensor is extracellular mitochondrial DNA.

In certain embodiments, disclosed herein are methods of augmenting and/or enhancing type I IFN production in vivo, through utilization of the cGAS-STING pathway. In some instances, also included herein is a method of activating and enhancing the cGAS-STING response. In some cases, the method comprises administering an inhibitor of a 2′3′-cGAMP degradation polypeptide. In additional cases, the methods comprise designing inhibitors of 2′3′-cGAMP degradation polypeptides and assays for evaluating the enzyme activity of the GMP degradation polypeptides.

cGAS-STING Pathway and the Production of Type I IFNs

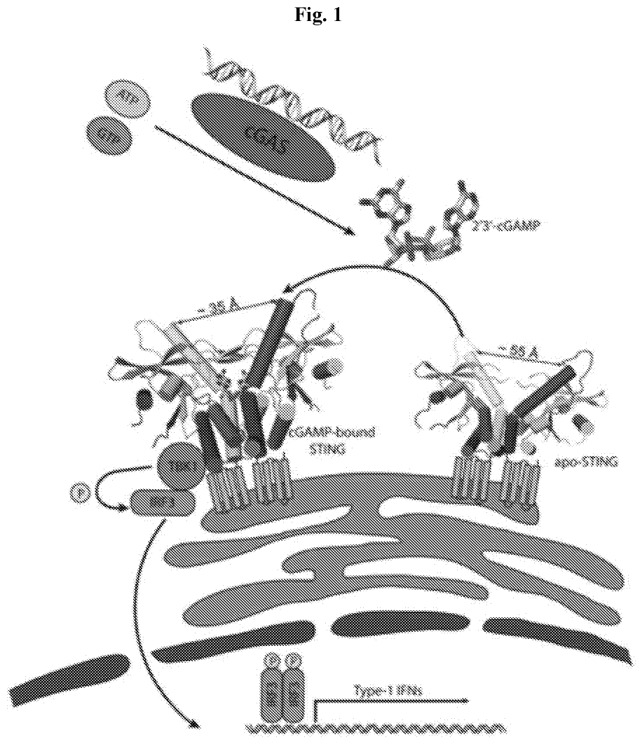

Cytosolic DNA can signal the presence of an infection within a cell or at a nearby cell. These cytosolic DNAs (e.g., double stranded DNAs) are surveyed by DNA sensors such as RNA pol III, DAI, IFI16, DDX41, LSm14A, cyclic-GMP-AMP synthase, LRRFIP1, Sox2, DHX9/36, Ku70 and AIM2. Cyclic-GMP-AMP synthase (cGAS or cGAMP synthase) is a 522 amino acid protein that belongs to the nucleotidyltransferase family of cytosolic DNA sensors. Upon cytosolic DNA stimulation, cGAS synthesizes cGAMP, which comprises a first bond between the 2′-OH of GMP and the 5′-phosphate of AMP and a second bond between the 3′-OH of AMP and the 5′-phosphate of GMP. cGAMP (also known as cyclic GMP-AMP, 2′3′-cGAMP, cGAMP (2′-5′) or cyclic Gp(2′-5′)Ap(3′-5′)) serves as a ligand to STING, thereby activating the STING-mediated IFN (e.g., IFNβ) production (FIG. 1).

STING (also known as stimulator of interferon genes, TMEM173, MITA, ERIS, or MPYS) is a 378 amino acid protein that comprises a N-terminal region containing four trans-membrane domains and a C-terminal domain that comprises a dimerization domain. Upon binding to 2′3′-cGAMP, STING undergoes a conformational rearrangement enclosing the 2′3′-cGAMP molecule.

Binding of 2′3′-cGAMP activates a cascade of events whereby STING recruits and activates IκB kinase (IKK) and TANK-binding kinase (TBK1), which following their phosphorylation, respectively activate nuclear transcription factor κB (NF-κB) and interferon regulatory factor 3 (IRF3). In some instances, the activated proteins translocate to the nucleus to induce transcription of the genes encoding type I IFN and cytokines for promoting intercellular host immune defense.

In some instances, STING is capable of directly sensing bacterial cyclic dinucleotides (CDNs) such as c[di-GMP]. In some cases, 2′3′-cGAMP acts as a second messenger binding to STING in response to cells exposed to cytosolic DNA.

### Pathogens

As described above, the presence of intracellular nucleic acid from a pathogen activates cGAS, leading to production of 2′3′-cGAMP, and subsequent activation of the STING pathway. In some instances, the pathogen is a virus, e.g., a DNA virus or an RNA virus. In some cases, the pathogen is a retrovirus. Exemplary viruses capable of subsequent activation of STING include, but are not limited to, herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV). In other instances, the pathogen is a bacterium. Exemplary bacteria include, but are not limited to, Listeria monocytogenes, Mycobacterium tuberculosis, Francisella novicida, Legionella pneumophila, Chlamydia trachomatis, Streptococcus pneumoniae, or Neisseria gonorrhoeae.

### Virus

In some embodiments, the pathogen is a DNA virus. In some instances, the DNA virus is a single-stranded DNA virus. In other instances, the DNA virus is a double-stranded DNA virus. In some cases, the virus utilizes a DNA-dependent DNA polymerase for replication.

In some embodiments, the pathogen is an RNA virus. In some instances, the RNA virus is a single-stranded RNA virus (e.g., single-stranded-positive sense or single-stranded-negative sense). In other instances, the RNA virus is a double-stranded RNA virus. Exemplary RNA viruses include vesicular stomatitis virus (VSV), Sendai virus, Hepatitis C virus, dengue fever virus, yellow fever virus, ebola virus, Marburg virus, venezuelan equine encephalitis virus, or zika virus. In some embodiments, the RNA virus is dengue fever virus, yellow fever virus, ebola virus, Marburg virus, venezuelan equine encephalitis virus, or zika virus.

In some embodiments, infection due to a viral pathogen induces release of mitochondrial DNA (mtDNA). In some embodiments, the release of mtDNA due to a viral pathogen induces a cGAS-induced antiviral response. In some instances, the viral pathogen is a ssRNA virus. In some instances, the viral pathogen is a dengue virus.

In some embodiments, the pathogen is a retrovirus. Retroviruses are single-stranded RNA viruses with a DNA intermediate. In most viruses, DNA is transcribed into RNA, and then RNA is translated into protein. However, retroviruses function differently, as their RNA is reverse-transcribed into DNA. Upon infection of a cell by a retrovirus, the retroviral RNA genome is transcribed into its corresponding double-stranded DNA by a reverse transcriptase enzyme which is coded for by the viral genome, which is the reverse of the usual pattern, thus retro (backwards). This DNA then enters the nucleus and integrates into the host DNA using an integrase enzyme which is also coded for by the viral genome. The integrated viral DNA (“proviral” DNA) becomes a component of the host genome, replicating with it and producing the proteins required in assembling new copies of the virus. It is difficult to detect the virus until it has infected the host. The information contained in a retroviral gene is thus used to generate the corresponding protein via the sequence: RNA→DNA→RNA→polypeptide.

The genome of a retrovirus (in either the RNA or DNA form) is divided conceptually into two parts. The first, or “trans-acting,” category consists of the regions coding for viral proteins. These include the group specific antigen (“gag”) gene for synthesis of the core coat proteins, the “pol” gene for the synthesis of various enzymes (such as reverse transcriptase), and the envelope (“env”) gene for the synthesis of envelope glycoproteins. The full length RNA transcript is packaged by the viral proteins into a viral particle which then buds off in a piece of cell membrane, in which are embedded env-derived peptides. This membrane-coated viral particle is a fully competent viral particle and go on to infect other cells.

In general, the second part of the retroviral genome is referred to as the “cis-acting” portion and consists of the regions which must be on the genome to allow its packaging and replication. This includes the packaging signal on an RNA molecule, such as the viral RNA, which identifies that RNA molecule to viral proteins as one to be encapsidated, Long Terminal Repeats (“LTRs”) with promoters and polyadenylation sites, and two start sites for DNA replication. The promoters, enhancers, and other regions of the LTRs are also capable of conferring tissue specificity such that the virus will only be “expressed” (i.e., transcribed and translated) in specific cell types even though it infects others.

### Exemplary Viruses

### Herpes Simplex Virus 1 (HSV-1)

HSV-1 is a highly contagious infection, which is common and endemic throughout the world. Most HSV-1 infections are acquired during childhood. The vast majority of HSV-1 infections are oral herpes (infections in or around the mouth, sometimes called orolabial, oral-labial or oral-facial herpes), but a proportion of HSV-1 infections are genital herpes (infections in the genital or anal area). HSV-1 is mainly transmitted by oral-to-oral contact to cause oral herpes infection, via contact with the HSV-1 virus in sores, saliva, and surfaces in or around the mouth. However, HSV-1 is also transmitted to the genital area through oral-genital contact to cause genital herpes.

### Murine Gamma-Herpesvirus 68 (MHV68)

MHV-68 is a rodent pathogen and a member of the gammaherpesvirus subfamily. MHV-68 has the ability to establish latent infections within lymphocytes and make close associations with cell tumors. MHV-68 establishes latency unless the host immune system is compromised, and this latency is regulated by multiple cellular controls, such as virus-specific open reading frames that result in gene products promoting the maintenance of latency or activation of lytic cycles. One of the major consequences of MHV-68 in mice is infectious mononucleosis. MHV-68 infection sites consist of primarily lung epithelial cells, adrenal glands, and heart tissue, with latent infection in B lymphocytes.

### Kaposi's Sarcoma-Associated Herpesvirus (KSHV)

KSHV or human herpesvirus 8 (HHV8) is a human rhadinovirus (gamma-2 herpesvirus) belonging to the family of herpesviruses. KSHV is a large double-stranded DNA virus with a protein covering that packages its nucleic acids, called the capsid, which is then surrounded by an amorphous protein layer called the tegument, and finally enclosed in a lipid envelope derived in part from the cell membrane. This virus is transmitted both sexually and through body fluids, for example, saliva and blood). KSHV causes a blood vessel cancer called Kaposi's sarcoma (KS), a lymphoma (a cancer of the lymphocyte) called body cavity-based lymphoma and some forms of severe lymph node enlargement, called Castleman's disease.

### Vaccinia Virus (VACV)

Vaccinia virus (VACV or VV) is a large, complex, enveloped virus belonging to the poxvirus family. The poxviruses are the largest known DNA viruses and are distinguished from other viruses by their ability to replicate entirely in the cytoplasm of infected cells. Poxviruses do not require nuclear factors for replication and, thus, replicate with little hindrance in enucleated cells. VACV has a linear, double-stranded DNA genome of approximately 190 kb in length, which encodes for around 250 genes. The genome is surrounded by a lipoprotein core membrane. Vaccinia virus is well-known for its role as a vaccine that eradicated the smallpox disease. The natural host of Vaccinia virus is unknown, but the virus replicates in cows and humans. During its replication cycle, Vaccinia virus produces four infectious forms which differ in their outer membranes: intracellular mature virion (IMV), the intracellular enveloped virion (IEV), the cell-associated enveloped virion (CEV) and the extracellular enveloped virion (EEV).

### Adenovirus

Adenoviruses are double-stranded DNA viruses and are now known to be a common cause of asymptomatic respiratory tract infection. An extremely hardy virus, adenovirus is ubiquitous in human and animal populations, survives long periods outside a host, and is endemic throughout the year. Possessing 52 serotypes, adenovirus is recognized as the etiologic agent of various diverse syndromes. It is transmitted via direct inoculation to the conjunctiva, a fecal-oral route, aerosolized droplets, or exposure to infected tissue or blood. The virus is capable of infecting multiple organ systems; however, most infections are asymptomatic.

### Human Papillomaviruses (HPV)

Human papillomaviruses (HPV), DNA virus from the papillomavirus family, are common viruses that cause warts. There are more than 100 types of HPV. Most are harmless, but about 30 types put you at risk for cancer. These types affect the genitals and you get them through sexual contact with an infected partner. They are either low-risk or high-risk. Low-risk HPV causes genital warts. High-risk HPV leads to cancers of the cervix, vulva, vagina, and anus in women and cancers of the anus and penis in men.

### Hepatitis B Virus (HBV)

HBV, a member of the Hepadnaviridae family, is a small DNA virus with unusual features similar to retroviruses. HBV replicates through an RNA intermediate and integrates into the host genome. Hepatitis B is one of a few known non-retroviral viruses which use reverse transcription as a part of its replication process. The features of the HBV replication cycle confer a distinct ability of the virus to persist in infected cells. HBV infection leads to a wide spectrum of liver disease ranging from acute (including fulminant hepatic failure) to chronic hepatitis, cirrhosis, and hepatocellular carcinoma. Acute HBV infection is either asymptomatic or presents with symptomatic acute hepatitis. About 5%-10% of population infected is unable to clear the virus and becomes chronically infected. Many chronically infected persons have mild liver disease. Other individuals with chronic HBV infection develop active disease, which progresses to cirrhosis and liver cancer.

### Hepatitis D Virus (HDV)

Hepatitis D virus (HDV) is a small spherical enveloped viroid. HDV is considered to be a subviral satellite because it can propagate only in the presence of the hepatitis B virus (HBV). Transmission of HDV can occur either via simultaneous infection with HBV (coinfection) or superimposed on chronic hepatitis B or hepatitis B carrier state (superinfection). Both superinfection and coinfection with HDV results in more severe complications compared to infection with HBV alone. These complications include a greater likelihood of experiencing liver failure in acute infections and a rapid progression to liver cirrhosis, with an increased risk of developing liver cancer in chronic infections. In combination with hepatitis B virus, hepatitis D has the highest fatality rate of all the hepatitis infections, at 20%.

### Human Immunodeficiency Virus (HIV)

The human immunodeficiency virus (HIV) is a lentivirus (a subgroup of retrovirus) that causes HIV infection and over time acquired immunodeficiency syndrome (AIDS). AIDS is a condition in humans in which progressive failure of the immune system allows life-threatening opportunistic infections and cancers to thrive. Infection with HIV occurs by the transfer of blood, semen, vaginal fluid, pre-ejaculate, or breast milk. Within these bodily fluids, HIV is present as both free virus particles and virus within infected immune cells. HIV infects vital cells in the human immune system such as helper T cells (specifically CD4⁺ T cells), macrophages, and dendritic cells. HIV infection leads to low levels of CD4⁺ T cells through a number of mechanisms, including but not limited to, pyroptosis of abortively infected T cells, apoptosis of uninfected bystander cells, direct viral killing of infected cells, and killing of infected CD4⁺ T cells by CD8 cytotoxic lymphocytes that recognize infected cells. When CD4⁺ T cell numbers decline below a critical level, cell-mediated immunity is lost, and the body becomes progressively more susceptible to opportunistic infections.

### Human Cytomegalovirus (HCMV)

Human cytomegalovirus (HCMV) is a beta-herpesvirus that causes lifelong infection in humans. HCMV has a prevalence of 55-100% within the human population. Primary HCMV infection is generally asymptomatic in healthy hosts but causes severe and sometimes fatal disease in immunocompromised individuals, organ transplant recipients, and neonates. HCMV is the leading infectious cause of congenital abnormalities in the Western world, affecting 1-2.5% of all live births. After infection, HCMV remains latent within the body throughout life and is reactivated at any time. Eventually, it causes mucoepidermoid carcinoma and other malignancies such as prostate cancer. Although they are found throughout the body, HCMV infections are frequently associated with the salivary glands. The mode of HCMV transmission from person to person is entirely unknown but is presumed to occur through bodily fluids. Infection requires close, intimate contact with a person secreting the virus in their saliva, urine, or other bodily fluids. HCMV is transmitted sexually and via breast milk, and also occurs through receiving transplanted organs or blood transfusions.

### Dengue Fever Virus

Dengue fever virus (DENV) is an RNA virus of the family Flaviviridae; genus Flavivirus. It is transmitted by arthropods (mosquitoes or ticks), and is therefore also referred to as a arbovirus (arthropod-borne virus). Dengue virus is primarily transmitted by Aedes mosquitoes, particularly A. aegypti. Other Aedes species that transmit the disease include A. albopictus, A. polynesiensis and A. scutellaris. Humans are the primary host of the virus but it also circulates in nonhuman primates. A female mosquito that takes a blood meal from a person infected with dengue fever, during the initial 2- to 10-day febrile period, becomes itself infected with the virus in the cells lining its gut. About 8-10 days later, the virus spreads to other tissues including the mosquito's salivary glands and is subsequently released into its saliva. The virus seems to have no detrimental effect on the mosquito, which remains infected for life.

### Yellow Fever Virus

Yellow fever virus is an RNA virus that belongs to the genus Flavivirus. It is related to West Nile, St. Louis encephalitis, and Japanese encephalitis viruses. Yellow fever virus is transmitted to people primarily through the bite of infected Aedes or Haemagogus species mosquitoes. Mosquitoes acquire the virus by feeding on infected primates (human or non-human) and then can transmit the virus to other primates (human or non-human). People infected with yellow fever virus are infectious to mosquitoes (referred to as being “viremic”) shortly before the onset of fever and up to 5 days after onset.

### Ebola Virus

Ebola virus (EBOV) is one of five known viruses within the genus Ebolavirus. Four of the five known ebolaviruses, including EBOV, cause a severe and often fatal hemorrhagic fever in humans and other mammals, known as Ebola virus disease (EVD). The EBOV genome is a single-stranded RNA approximately 19,000 nucleotides long. It encodes seven structural proteins: nucleoprotein (NP), polymerase cofactor (VP35), (VP40), GP, transcription activator (VP30), VP24, and RNA-dependent RNA polymerase (L).

### Marburg Virus

Marburg virus is a hemorrhagic fever virus of the Filoviridae family of viruses and a member of the species Marburg marburgvirus, genus Marburgvirus. Marburg virus (MARV) causes Marburg virus disease in humans and nonhuman primates, a form of viral hemorrhagic fever. The virus is considered to be extremely dangerous.

### Zika Virus

Zika virus (ZIKV) is a member of the virus family Flaviviridae. It is spread by daytime-active Aedes mosquitoes, such as A. aegypti and A. albopictus. Zika virus is related to the dengue, yellow fever, Japanese encephalitis, and West Nile viruses. Since the 1950s, it has been known to occur within a narrow equatorial belt from Africa to Asia. The infection, known as Zika fever or Zika virus disease, often causes no or only mild symptoms, similar to a very mild form of dengue fever. While there is no specific treatment, paracetamol (acetaminophen) and rest may help with the symptoms. Zika can spread from a pregnant woman to her baby. This can result in microcephaly, severe brain malformations, and other birth defects. Zika infections in adults may result rarely in Guillain-Barré syndrome.

### Venezuelan Equine Encephalitis Virus (VEEV)

Venezuelan equine encephalitis virus is a member of the virus family Togaviridae, genus Alphavirus. Venezuelan equine encephalitis virus is a mosquito-borne viral pathogen that causes Venezuelan equine encephalitis or encephalomyelitis (VEE). VEE can affect all equine species, such as horses, donkeys, and zebras. After infection, equines may suddenly die or show progressive central nervous system disorders. Humans also can contract this disease. Healthy adults who become infected by the virus may experience flu-like symptoms, such as high fevers and headaches. People with weakened immune systems and the young and the elderly can become severely ill or die from this disease.

The virus that causes VEE is transmitted primarily by mosquitoes that bite an infected animal and then bite and feed on another animal or human. The speed with which the disease spreads depends on the subtype of the VEE virus and the density of mosquito populations. Enzootic subtypes of VEE are diseases endemic to certain areas. Generally these serotypes do not spread to other localities. Enzootic subtypes are associated with the rodent-mosquito transmission cycle. These forms of the virus can cause human illness but generally do not affect equine health. Epizootic subtypes, on the other hand, can spread rapidly through large populations. These forms of the virus are highly pathogenic to equines and can also affect human health.

### Bacteria

In some embodiments, a pathogen described herein is a bacterium. Bacteria are microscopic single-celled microorganisms that exist either as independent (free-living) organisms or as parasites (dependent on another organism for life) and thrive in diverse environments. As prokaryotes, the organism consists of a single cell with a simple internal structure. Bacterial DNA floats free, in a twisted thread-like mass called the nucleoid. Bacterial cells also contain separate, circular pieces of DNA called plasmids. Bacteria lack membrane-bound organelles, specialized cellular structures that are designed to execute a range of cellular functions from energy production to the transport of proteins. However, both bacterial cells contain ribosomes. A few different criteria are used to classify bacteria. They are distinguished by the nature of their cell walls, by their shape, or by differences in their genetic makeup.

### Exemplary Bacteria

Listeria monocytogenes

Listeria monocytogenes is the species of pathogenic bacteria that causes the infection listeriosis. L monocytogenes is a motile, non-spore-forming, gram-positive bacillus that has aerobic and facultative anaerobic characteristics making it capable of surviving in the presence or absence of oxygen. It grows best at neutral to slightly alkaline pH and is capable of growth at a wide range of temperatures, from 1-45° C. It is beta-hemolytic and has a blue-green sheen on blood-free agar. It exhibits characteristic tumbling motility when viewed with light microscopy. It grows and reproduces inside the host's cells and is one of the most virulent foodborne pathogens, with 20 to 30% of food borne listeriosis infections in high-risk individuals are fatal. Most infections occur after oral ingestion, with access to the systemic circulation after intestinal penetration. CNS infection manifests as meningitis, meningoencephalitis, or abscess. Endocarditis is another possible presentation. Localized infection manifests as septic arthritis, osteomyelitis, and, rarely, pneumonia.

Mycobacterium tuberculosis

Mycobacterium tuberculosis is an obligate pathogenic bacterial species in the family Mycobacteriaceae and the causative agent of tuberculosis. M. tuberculosis has an unusual, waxy coating on its cell surface (primarily due to the presence of mycolic acid), which makes the cells impervious to Gram staining. The physiology of M. tuberculosis is highly aerobic and requires high levels of oxygen. Primarily a pathogen of the mammalian respiratory system, it infects the lungs.

Francisella novicida

Francisella novicida is a bacterium of the Francisellaceae family, which consist of Gram-negative pathogenic bacteria. These bacteria vary from small cocci to rod-shaped, and are most known for their intracellular parasitic capabilities. Some of the main symptoms associated with this infection include pneumonia, muscle pain, and fever.

Legionella pneumophila

Legionella pneumophila is a thin, aerobic, pleomorphic, flagellated, nonspore-forming, Gram-negative bacterium of the genus Legionella. L. pneumophila infection causes Legionnaires' disease, a severe form of pneumonia. The symptoms of Legionnaire's disease include confusion, headache, diarrhoea, abdominal pain, fever, chills, and myalgia as well as a non-productive cough. Pontiac fever is a non-pneumonic form of L. pneumophila infection. Symptoms are flu-like, including fever, tiredness, myalgia, headache, sore throat, nausea, and sometimes cough. L. pneumophila is transmitted by aerosols and aspiration of contaminated water.

Chlamydia trachomatis

Chlamydia trachomatis is a gram-negative bacterium that infects the columnar epithelium of the cervix, urethra, and rectum, as well as nongenital sites such as the lungs and eyes. The bacterium is the cause of the most frequently reported sexually transmitted disease in the United States. Most persons with this infection are asymptomatic. Untreated infection results in serious complications such as pelvic inflammatory disease, infertility, and ectopic pregnancy in women, and epididymitis and orchitis in men. Men and women experience chlamydia-induced reactive arthritis. In neonates and infants, the bacterium causes conjunctivitis and pneumonia. Adults also experience conjunctivitis caused by chlamydia. Trachoma is a recurrent ocular infection caused by chlamydia.

Streptococcus pneumoniae

Streptococcus pneumoniae, or pneumococcus is a Gram-positive, alpha-hemolytic (under aerobic conditions) or beta-hemolytic (under anaerobic conditions), facultative anaerobic member of the genus Streptococcus, that is responsible for the majority of community-acquired pneumonia. It is a commensal organism in the human respiratory tract, meaning that it benefits from the human body, without harming it. However, infection by pneumococcus is dangerous, causing not only pneumonia, but also bronchitis, otitis media, septicemia, and meningitis. Pneumococcal pneumonia causes fever and chills, coughs, difficulty breathing, and chest pain. If the infection spreads to the brain and spinal cord, it causes pneumococcal meningitis, characterized by a stiff neck, fever, confusion, and headaches. S. pneumoniae primarily spreads through the air in the form of aerosol droplets from coughing and sneezing.

Neisseria gonorrhoeae

Neisseria gonorrhoeae, also known as gonococci (plural), or gonococcus (singular), is a species of Gram-negative, fastidious, coffee bean-shaped diplococci bacteria responsible for the sexually transmitted infection gonorrhea. Neisseria gonorrhoeae grow and rapidly multiply in the mucous membranes, especially the mouth, throat, and anus of males and females, and the cervix, fallopian tubes, and uterus of the female reproductive tract. N. gonorrhoeae is transmitted from person to person via oral, vaginal, and anal sexual contact. During childbirth, infants contract the infection in the birth canal resulting in bilateral conjunctivitis.

### Phosphodiesterases

Phosphodiesterases comprise a class of enzymes that catalyze the hydrolysis of a phosphodiester bond. In some instances, phosphodiesterase has been linked with viral infection and its inhibition has been correlated with a reduction in viral replication. In some instances, the class of phosphodiesterases further comprises cyclic nucleotide phosphodiesterases, phospholipases C and D, autotaxin, sphingomyelin phosphodiesterase, DNases, RNases, restriction endonucleases, and small-molecule phosphodiesterases.

Cyclic nucleotide phosphodiesterases (PDEs) regulate the cyclic nucleotides cAMP and cGMP. In some instances, cAMP and cGMP function as intracellular second messengers to transduce a variety of extracellular signals including hormones, light, and neurotransmitters. In some cases, PDEs degrade cyclic nucleotides to their corresponding monophosphates, thereby regulating the intracellular concentrations of cyclic nucleotides and their effects on signal transduction.

In some embodiments, PDEs are classified into classes I, II and III. In some cases, mammalian PDEs, which belong to Class I PDEs, are further divided into 12 families (PDE1-PDE12) based on their substrate specificity and affinity, sensitivity to cofactors, and sensitivity to inhibitory agents. In some cases, the different families of mammalian PDEs further contain splice variants that can be unique in tissue-expression patterns, gene regulation, enzymatic regulation by phosphorylation and regulatory proteins, subcellular localization, and interaction with association proteins.

PDE1 family comprises Ca²⁺/calmodulin-dependent PDEs. In some cases, PDE1 is encoded by at least three different genes, each having at least two different splice variants, PDE1A and PDE1B. In some cases, PDE1 isozymes are regulated in vitro by phosphorylation/dephosphorylation. For example, phosphorylation decreases the affinity of PDE for calmodulin, decreases the activity of PDE1, and increases steady state levels of cAMP. In some cases, PDE1 is observed in the lung, heart, and brain.

PDE2s are cGMP-stimulated PDEs that have been observed in the cerebellum, neocortex, heart, kidney, lung, pulmonary artery, and skeletal muscle. In some cases, PDE2 mediates the effects of cAMP on catecholamine secretion, participate in the regulation of aldosterone, and play a role in olfactory signal transduction.

The family of PDE3s has a high affinity for both cGMP and cAMP. PDE3 plays a role in stimulating myocardial contractility, inhibiting platelet aggregation, relaxing vascular and airway smooth muscle, inhibiting proliferation of T-lymphocytes and cultured vascular smooth muscle cells, and regulating catecholamine-induced release of free fatty acids from adipose tissue. In some instances, isozymes of PDE3 are regulated by cAMP-dependent protein kinase, or by insulin-dependent kinases.

In some embodiments, PDE4s are specific for cAMP and are activated by cAMP-dependent phosphorylation. In some cases, PDE4s are localized to airway smooth muscle, the vascular endothelium, and all inflammatory cells.

PDE5s exert selective recognition for cGMP as a substrate, and comprise two allosteric cGMP-specific binding sites. In some cases, binding of cGMP to these allosteric binding sites modulate phosphorylation of PDE5 by cGMP-dependent protein kinase. In some cases, elevated levels of PDE5 are found in vascular smooth muscle, platelets, lung, and kidney.

PDE6s, the photoreceptor cyclic nucleotide phosphodiesterases, are involved in the phototransduction cascade. In association with the G-protein transducin, PDE6s hydrolyze cGMP to regulate cGMP-gated cation channels in photoreceptor membranes. In addition to the cGMP-binding active site, PDE6s also have two high-affinity cGMP-binding sites which may further play a regulatory role in PDE6 function.

The PDE7 family of PDEs is cAMP specific and comprises one known member having multiple splice variants. Although mRNAs encoding PDE7s are found in skeletal muscle, heart, brain, lung, kidney, and pancreas, expression of PDE7 proteins is restricted to specific tissue types. Further, PDE7s shares a high degree of homology to the PDE4 family.

PDE8s are cAMP specific, and similar to PDE7, are closely related to the PDE4 family. In some cases, PDE8s are expressed in thyroid gland, testis, eye, liver, skeletal muscle, heart, kidney, ovary, and brain.

PDE9s are cGMP specific and closely resemble the PDE8 family of PDEs. In some cases, PDE9s are expressed in kidney, liver, lung, brain, spleen, and small intestine.

PDE10s are dual-substrate PDEs, hydrolyzing both cAMP and cGMP. In some instances, PDE10s are expressed in brain, thyroid, and testis.

PDE11s, similar to PDE10s, are dual-substrate PDEs that hydrolyze both cAMP and cGMP. In some instances, PDE11s are expressed in the skeletal muscle, brain, lung, spleen, prostate gland, and testis.

PDE12s hydrolyze cAMP and oligoadenylates (e.g., 2′,5′-oligoadenylate). In some cases, although PDE12 hydrolyzes the 2′5′ linkage, PDE12 does not exhibit activity toward 2′3′-cGAMP.

### Ecto-Nucleotide Pyrophosphatase/Phosphodiesterase

In some embodiments, the class of phosphodiesterases also comprises an ecto-nucleotide pyrophosphatase/phosphodiesterase. Ecto-nucleotide pyrophosphatase/phosphodiesterases (ENPP) or nucleotide pyrophosphatase/phosphodiesterases (NPP) are a subfamily of ectonucleotidases which hydrolyze the pyrophosphate and phosphodiester bonds of their substrates to nucleoside 5′-monophosphates. In some embodiments, ENPP (or NPP) comprises seven members, ENPP-1, ENPP-2, ENPP-3, ENPP-4, ENPP-5, ENPP-6 and ENPP-7.

The ecto-nucleotide pyrophosphatase/phosphodiesterase 1 (ENPP-1) protein (also known as PC-1) is a type II transmembrane glycoprotein comprising two identical disulfide-bonded subunits. In some instances, ENPP-1 is expressed in precursor cells and promotes osteoblast differentiation and regulates bone mineralization. In some instances, ENPP-1 negatively regulates bone mineralization by hydrolyzing extracellular nucleotide triphosphates (NTPs) to produce inorganic pyrophosphate (PPi). In some cases, expression of ENPP-1 has been observed in pancreas, kidney, bladder, and the liver.

In some embodiments, ENPP-1 has a broad specificity and cleaves a variety of substrates, including phosphodiester bonds of nucleotides and nucleotide sugars and pyrophosphate bonds of nucleotides and nucleotide sugars. In some instances, ENPP-1 functions to hydrolyze nucleoside 5′ triphosphates to their corresponding monophosphates and also hydrolyze diadenosine polyphosphates. In some cases, ENPP-1 hydrolyzes the 2′5′ linkage of cyclic nucleotides. In some cases, ENPP-1 degrades 2′3′-cGAMP, a substrate of STING.

In some embodiments, ENPP-1 comprises two N-terminal somatomedin B (SMB)-like domains (SMB1 and SMB2), a catalytic domain and a C-terminal nuclease-like domain. In some cases, the two SMB domains is connected to the catalytic domain by a first flexible linker, while the catalytic domain is further connected to the nuclease-like domain by a second flexible linker. In some instances, the SMB domains facilitate ENPP-1 dimerization. In some cases, the catalytic domain comprises the NTP binding site. In some cases, the nuclease-like domain comprises an EF-hand motif, which binds Ca⁺² ion.

In some cases, ENPP-2 and ENPP-3 are type II transmembrane glycoproteins that share a similar architecture with ENPP-1, for example, comprising the two N-terminal SMB-like domains, a catalytic domain, and a nuclease-like domain. In some instances, ENPP-2 hydrolyzes lysophospholipids to produce lysophosphatidic acid (LPA) or sphingosylphosphorylcholine (SPC) to produce sphingosine-1 phosphate (S1P). In some cases, ENPP-3 is identified to regulate N-acetylglucosaminyltransferase GnT-IX (GnT-Vb).

In some embodiments, ENPP-4-ENPP-7 are shorter proteins compared to ENPP-1-ENPP-3 and comprise a catalytic domain and lack the SMB-like and nuclease-like domains. ENPP-6 is a choline-specific glycerophosphodiesterase, with lysophospholipase C activity towards lysophosphatidylcholine (LPC). ENPP-7 is an alkaline sphingomyelinase (alk-SMase) with no detectable nucleotidase activity.

Inhibitor of 2′3′-cGAMP Degradation Polypeptide

In some embodiments, disclosed herein include an inhibitor of a 2′3′-cGAMP degradation polypeptide. In some instances, a 2′3′-cGAMP degradation polypeptide comprises a PDE protein. In some instances, a 2′3′-cGAMP degradation polypeptide comprises a PDE5 protein. In some instances, a 2′3′-cGAMP degradation polypeptide comprises a PDE10 protein. In some instances, a 2′3′-cGAMP degradation polypeptide comprises a Pan-PDE protein. In some cases, a 2′3′-cGAMP degradation polypeptide comprises a ENPP-1 protein. In some cases, an inhibitor of a 2′3′-cGAMP degradation polypeptide is a small molecule inhibitor. In some cases, an inhibitor of a 2′3′-cGAMP degradation polypeptide comprises a PDE5 inhibitor. In some cases, an inhibitor of a 2′3′-cGAMP degradation polypeptide comprises a PDE10 inhibitor. In some cases, an inhibitor of a 2′3′-cGAMP degradation polypeptide comprises a Pan-PDE inhibitor. In some cases, an inhibitor of a 2′3′-cGAMP degradation polypeptide comprises an ENPP-1 inhibitor.

In some embodiments, an inhibitor of a 2′3′-cGAMP degradation polypeptide (e.g., a ENPP-1 inhibitor) described herein is a reversible inhibitor. Reversible inhibitor interacts with an enzyme with non-covalent interactions, e.g., hydrogen bonds, hydrophobic interactions, and/or ionic bonds. In some instances, a reversible inhibitor is further classified as a competitive inhibitor, an allosteric inhibitor, or a mixed inhibitor. In competitive inhibition, both the inhibitor and the substrate compete for the same active site. In allosteric inhibition, the inhibitor binds to the enzyme at a non-active site which modulates the enzyme's activity but does not affect binding of the substrate. In some cases, an inhibitor of a 2′3′-cGAMP degradation polypeptide (e.g., a ENPP-1 inhibitor) described herein is a competitive inhibitor. In other cases, an inhibitor of a 2′3′-cGAMP degradation polypeptide (e.g., a ENPP-1 inhibitor) described herein is an allosteric inhibitor. In some cases, an inhibitor of a 2′3′-cGAMP degradation polypeptide (e.g., a ENPP-1 inhibitor) described herein is a mixed inhibitor. In some instances, a ENPP-1 inhibitor described herein is a competitive inhibitor. In other instances, a ENPP-1 inhibitor described herein is an allosteric inhibitor. In other instances, a ENPP-1 inhibitor described herein is a mixed inhibitor.

In some embodiments, an inhibitor of a 2′3′-cGAMP degradation polypeptide (e.g., a ENPP-1 inhibitor) described herein is an irreversible inhibitor. Irreversible inhibitor interacts with an enzyme with covalent interaction. In some cases, a ENPP-1 is an irreversible inhibitor.

In some embodiments, an inhibitor of a 2′3′-cGAMP degradation polypeptide (e.g., a ENPP-1 inhibitor) binds to one or more domains of a PDE described herein. In some cases, a PDE inhibitor binds to one or more domains of ENPP-1. As described above, ENPP-1 comprises a catalytic domain and a nuclease-like domain. In some instances, an inhibitor of a 2′3′-cGAMP degradation polypeptide (e.g., a ENPP-1 inhibitor) binds to the catalytic domain of ENPP-1. In some cases, an inhibitor of a 2′3′-cGAMP degradation polypeptide (e.g., a ENPP-1 inhibitor) binds to the nuclease-like domain of ENPP-1.

In some cases, an inhibitor of a 2′3′-cGAMP degradation polypeptide (e.g., a ENPP-1 inhibitor) selectively binds to a region on PDE (e.g., ENPP-1) also recognized by GMP. In some cases, an inhibitor of a 2′3′-cGAMP degradation polypeptide (e.g., a ENPP-1 inhibitor) selectively binds to a region on PDE (e.g., ENPP-1) also recognized by GMP but interacts weakly with the region that is bound by AMP. In some instances, an inhibitor of a 2′3′-cGAMP degradation polypeptide (e.g., a ENPP-1 inhibitor) does not inhibit the ATP hydrolysis function of PDE.

In some embodiments, an inhibitor of a 2′3′-cGAMP degradation polypeptide (e.g., a ENPP-1 inhibitor) comprises a di-adenosine pentaphosphate analogue, an ATP analogue, an oxadiazole derivative, a biscoumarine derivative, or a combination. In some instances, an inhibitor of a 2′3′-cGAMP degradation polypeptide (e.g., a ENPP-1 inhibitor) comprises a compound, its analogue, or its derivative as illustrated in Scheme I.

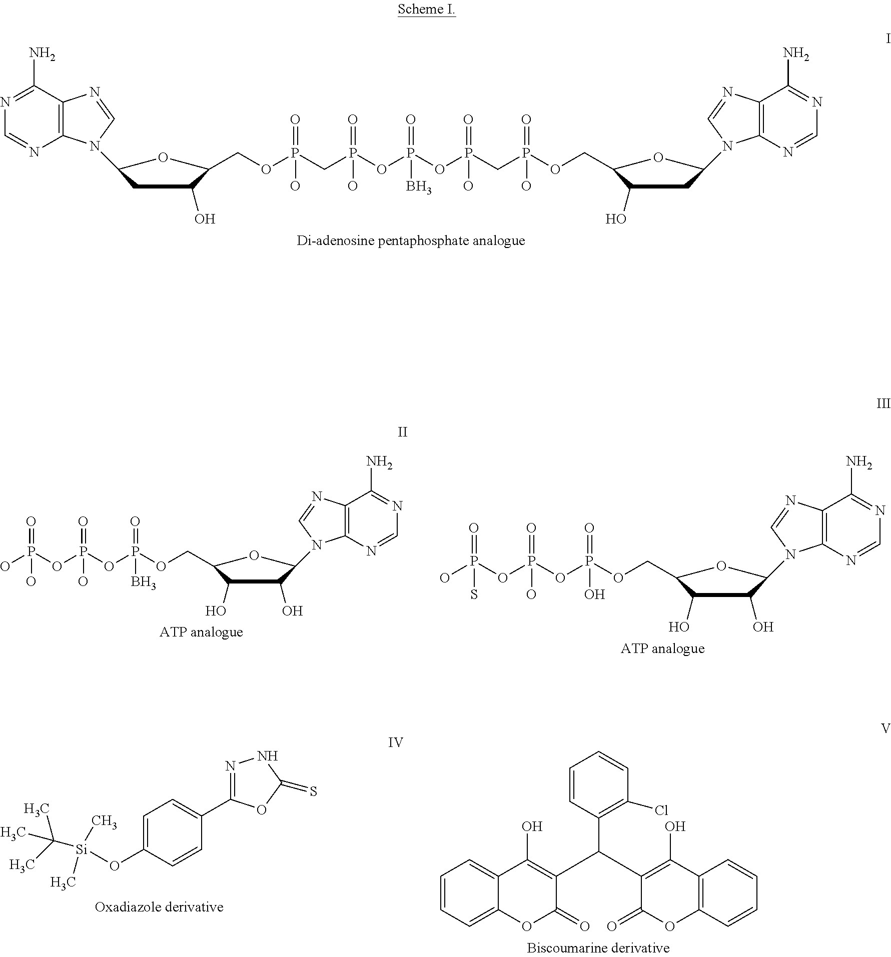

In some embodiments, an inhibitor of a 2′3′-cGAMP degradation polypeptide (e.g., a ENPP-1 inhibitor) comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, an inhibitor of a 2′3′-cGAMP degradation polypeptide (e.g., a ENPP-1 inhibitor) is ARL67156:

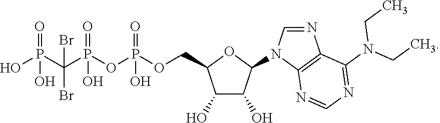 [SMILES: CCN(CC)c1ncnc2c1ncn2[C@@H]1O[C@H](COP(=O)(O)OP(=O)(O)C(Br)(Br)P(=O)(O)O)[C@@H](O)[C@H]1O]

In some instances, an inhibitor of a 2′3′-cGAMP degradation polypeptide (e.g., a ENPP-1 inhibitor) is diadenosine 5′,5″-boranopolyphosphonate:

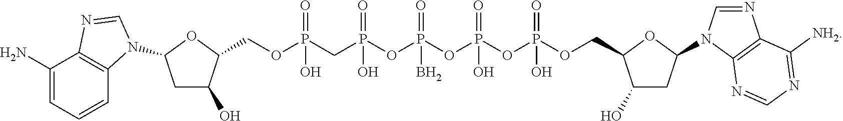

In some instances, an inhibitor of a 2′3′-cGAMP degradation polypeptide (e.g., a ENPP-1 inhibitor) is adenosine 5′-(α-borano)-β,γ-methylene triphosphate:

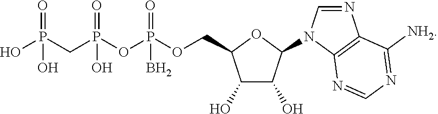

In some instances, an inhibitor of a 2′3′-cGAMP degradation polypeptide (e.g., a ENPP-1 inhibitor) is adenosine 5′-(γ-thio)-α,β-methylene triphosphate:

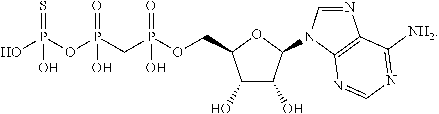 [SMILES: Nc1ncnc2c1ncn2[C@@H]1O[C@H](COP(=O)(O)CP(=O)(O)OP(O)(O)=S)[C@@H](O)[C@H]1O]

In some instances, an inhibitor of a 2′3′-cGAMP degradation polypeptide (e.g., a ENPP-1 inhibitor) is an oxadiazole derivative:

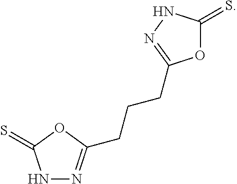 [SMILES: S=c1[nH]nc(CCCc2n[nH]c(=S)o2)o1]

In some instances, an inhibitor of a 2′3 ‘-cGAMP degradation polypeptide (e.g., a ENPP-1 inhibitor) is a biscoumarine derivative:

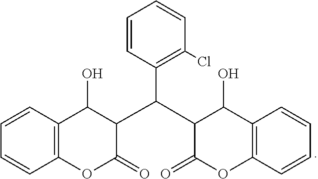 [SMILES: O=C1Oc2ccccc2C(O)C1C(c1ccccc1Cl)C1C(=O)Oc2ccccc2C1O]

In some instances, an inhibitor of a 2′3’-cGAMP degradation polypeptide (e.g., a ENPP-1 inhibitor) is reactive blue 2:

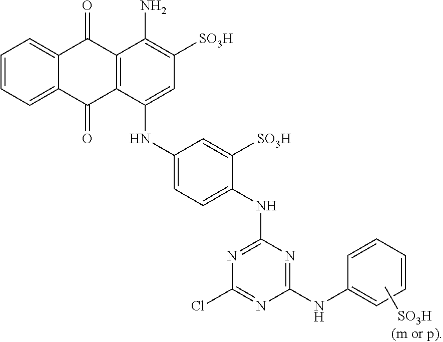

In some instances, an inhibitor of a 2′3 ‘-cGAMP degradation polypeptide (e.g., a ENPP-1 inhibitor) is suramin:

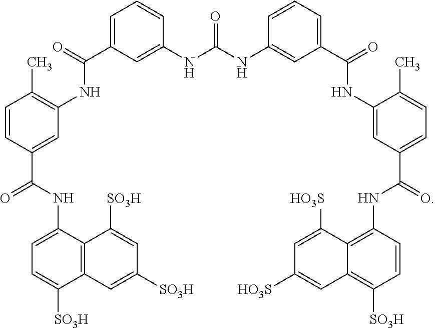 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

In some instances, an inhibitor of a 2′3’-cGAMP degradation polypeptide (e.g., a ENPP-1 inhibitor) is a quinazoline-4-piperidine-4-ethylsulfamide derivative:

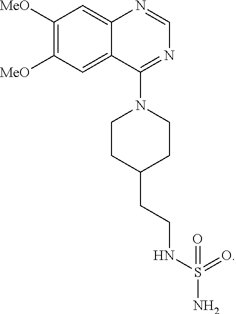 [SMILES: COc1cc2ncnc(N3CCC(CCNS(N)(=O)=O)CC3)c2cc1OC]

In some instances, an inhibitor of a 2′3′-cGAMP degradation polypeptide (e.g., a ENPP-1 inhibitor) is a thioacetamide derivative:

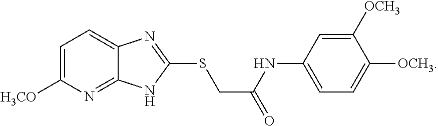 [SMILES: COc1ccc2nc(SCC(=O)Nc3ccc(OC)c(OC)c3)[nH]c2n1]

In some instances, an inhibitor of a 2′3′-cGAMP degradation polypeptide (e.g., a ENPP-1 inhibitor) is PSB-POM141:
[symbol] (a Keggin-type inorganic complex).

In some embodiments, an inhibitor of a 2′3′-cGAMP degradation polypeptide (e.g., a ENPP-1 inhibitor) comprises 2-(3H-imidazo[4,5-b]pyridin-2-ylthio)-N-(3,4-dimethoxyphenyl)acetamide or a derivative, analog, or salt thereof.

In some embodiments, an inhibitor of a 2′3′-cGAMP degradation polypeptide (e.g., a ENPP-1 inhibitor) comprises 2-(6-Amino-9H-purin-8-ylthio)-N-(3,4-dimethoxyphenyl)-acetamide, or a salt thereof.

In some embodiments, an inhibitor of a 2′3′-cGAMP degradation polypeptide (e.g., a ENPP-1 inhibitor) comprises N-(3,4-Dimethoxyphenyl)-2-(5-methoxy-3H-imidazo[4,5-b]-pyridin-2-ylthio)acetamide or a salt thereof.

In some embodiments, an inhibitor of a 2′3′-cGAMP degradation polypeptide (e.g., a ENPP-1 inhibitor) comprises 2-(1-(6,7-Dimethoxyquinazolin-4-yl)piperidin-4-yl)ethyl sulfamide or a salt thereof.

In some embodiments, an inhibitor of a 2′3′-cGAMP degradation polypeptide (e.g., a ENPP-1 inhibitor) comprises ((1-(6,7-Dimethoxyquinazolin-4-yl)piperidin-4-yl)methyl)sulfamide or a salt thereof.

In some embodiments, an inhibitor of a 2′3′-cGAMP degradation polypeptide (e.g., a ENPP-1 inhibitor) comprises SK4A (SAT0037) or a derivative or salt thereof.

In some instances, an inhibitor of a 2′3′-cGAMP degradation polypeptide (e.g., a ENPP-1 inhibitor) comprises a PDE inhibitor described in Chang, et al., “Imidazopyridine- and purine-thioacetamide derivatives: potent inhibitors of nucleotide pyrophosphatase/phosphodiesterase I (NPP1),” J. of Med. Chem., 57:10080-10100 (2014).

In some embodiments, an inhibitor of a 2′3′-cGAMP degradation polypeptide (e.g., a ENPP-1 inhibitor) comprises a PDE inhibitor described in Lee, et al., “Thiazolo[3,2-α]benzimidazol-3(2H)-one derivatives: structure-activity relationships of selective nucleotide pyrophosphatase/phosphodiesterase1 (NPP1) inhibitors,” Bioorganic & Medicinal Chemistry, 24:3157-3165 (2016).

In some embodiments, an inhibitor of a 2′3′-cGAMP degradation polypeptide (e.g., a ENPP-1 inhibitor) comprises a PDE inhibitor described in Shayhidin, et al., “Quinazoline-4-piperidine sulfamides are specific inhibitors of human NPP1 and prevent pathological mineralization of valve interstitial cells,” British Journal of Pharmacology, 172:4189-4199 (2015).

In some embodiments, an inhibitor of a 2′3′-cGAMP degradation polypeptide (e.g., a ENPP-1 inhibitor) comprises a PDE inhibitor described in Li, et al., “Hydrolysis of 2′3′-cGAMP by ENPP-1 and design of nonhydrolyzable analogs,” Nature Chemical Biology, 10:1043-1048 (2014).

In some embodiments, an inhibitor of a 2′3′-cGAMP degradation polypeptide (e.g., a ENPP-1 inhibitor) comprises Compound 1:

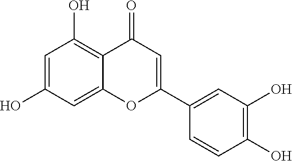 [SMILES: O=c1cc(-c2ccc(O)c(O)c2)oc2cc(O)cc(O)c12]

or a derivative, analog, or salt thereof.

In some embodiments, an inhibitor of a 2′3′-cGAMP degradation polypeptide (e.g., a ENPP-1 inhibitor) comprises Compound 2:

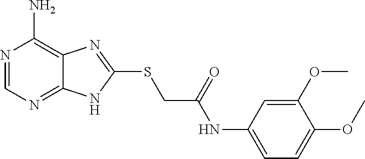 [SMILES: COc1ccc(NC(=O)CSc2nc3c(N)ncnc3[nH]2)cc1OC]

or a derivative, analog, or salt thereof.

In some embodiments, an inhibitor of a 2′3′-cGAMP degradation polypeptide (e.g., a ENPP-1 inhibitor) comprises Compound 3:

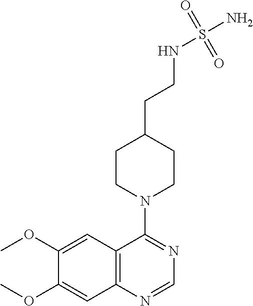 [SMILES: COc1cc2ncnc(N3CCC(CCNS(N)(=O)=O)CC3)c2cc1OC]

or a derivative, analog, or salt thereof.

### Methods of Use

In some embodiments, disclosed herein are methods of treating a subject having a pathogenic infection. In some instances, the method comprises administering to the subject an inhibitor of a 2′3′-cGAMP degradation polypeptide, wherein the inhibitor prevents hydrolysis of 2′3′-cGAMP and wherein the subject has an infection.

In some embodiments, the 2′3′-cGAMP degradation polypeptide is a phosphodiesterase (PDE). In some embodiments, the PDE comprises a cyclic nucleotide phosphodiesterase described supra. In some embodiments, the PDE comprises a PDE5 protein. In some cases, the PDE comprises a PDE10 protein. In some cases, the PDE comprises a Pan-PDE protein. In some embodiments, the PDE comprises an ectonucleotide pyrophosphatase/phosphodiesterase (ENPP) protein. In some cases, the ENPP protein comprises ectonucleotide pyrophosphatase/phosphodiesterase family member 1 (ENPP-1).

In some instances, a PDE inhibitor described herein comprises a small molecule. In some cases, the PDE inhibitor is a PDE5 inhibitor. In some cases, the PDE inhibitor is a PDE10 inhibitor. In some cases, the PDE inhibitor is a Pan-PDE inhibitor. In some cases, the PDE inhibitor is an ENPP-1 inhibitor.

In some embodiments, a PDE inhibitor described herein is a reversible inhibitor. In some instances, a reversible inhibitor is further classified as a competitive inhibitor or an allosteric inhibitor. In some cases, a PDE inhibitor described herein is a competitive inhibitor. In other cases, a PDE inhibitor described herein is an allosteric inhibitor. In some cases, a PDE inhibitor described herein is a mixed inhibitor. In some instances, a ENPP-1 inhibitor described herein is a competitive inhibitor. In other instances, a ENPP-1 inhibitor described herein is an allosteric inhibitor. In some instances, a ENPP-1 inhibitor described herein is a mixed inhibitor.

In some embodiments, a PDE inhibitor described herein is an irreversible inhibitor. In some cases, a ENPP-1 is an irreversible inhibitor.

In some embodiments, a PDE inhibitor binds to one or more domains of a PDE described herein. In some cases, a PDE inhibitor binds to one or more domains of ENPP-1. As described above, ENPP-1 comprises a catalytic domain and a nuclease-like domain. In some instances, a PDE inhibitor binds to the catalytic domain of ENPP-1. In some cases, a PDE inhibitor binds to the nuclease-like domain of ENPP-1.

In some cases, a PDE inhibitor selectively binds to a region on PDE (e.g., ENPP-1) also recognized by GMP. In some cases, a PDE inhibitor selectively binds to a region on PDE (e.g., ENPP-1) also recognized by GMP but interacts weakly with the region that is bound by AMP.

In some embodiments, a PDE inhibitor comprises a di-adenosine pentaphosphate analogue, an ATP analogue, an oxadiazole derivative, a biscoumarine derivative, or a combination. In some instances, a PDE inhibitor comprises a compound, its analogue, or its derivative as illustrated in Scheme I.

In some embodiments, a PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141. In some instances, a PDE inhibitor is ARL67156. In some instances, a PDE inhibitor is diadenosine 5′,5″-boranopolyphosphonate. In some instances, a PDE inhibitor is adenosine 5′-(α-borano)-β,γ-methylene triphosphate. In some instances, a PDE inhibitor is adenosine 5′-(γ-thio)-α,β-methylene triphosphate. In some instances, a PDE inhibitor is an oxadiazole derivative. In some instances, a PDE inhibitor is a biscoumarine derivative. In some instances, a PDE inhibitor is reactive blue 2. In some instances, a PDE inhibitor is suramin. In some instances, a PDE inhibitor is a quinazoline-4-piperidine-4-ethylsulfamide derivative. In some instances, a PDE inhibitor is a thioacetamide derivative. In some instances, a PDE inhibitor is PSB-POM141 (a Keggin-type inorganic complex).

In some embodiments, a PDE inhibitor comprises 2-(3H-imidazo[4,5-b]pyridin-2-ylthio)-N-(3,4-dimethoxyphenyl)acetamide or a derivative, analog, or salt thereof.

In some embodiments, a PDE inhibitor comprises 2-(6-Amino-9H-purin-8-ylthio)-N-(3,4-dimethoxyphenyl)-acetamide, or a salt thereof.

In some embodiments, a PDE inhibitor comprises N-(3,4-Dimethoxyphenyl)-2-(5-methoxy-3H-imidazo[4,5-b]-pyridin-2-ylthio)acetamide or a salt thereof.

In some embodiments, a PDE inhibitor comprises 2-(1-(6,7-Dimethoxyquinazolin-4-yl)piperidin-4-yl)ethyl sulfamide or a salt thereof.

In some embodiments, a PDE inhibitor comprises ((1-(6,7-Dimethoxyquinazolin-4-yl)piperidin-4-yl)methyl)sulfamide or a salt thereof.

In some embodiments, a PDE inhibitor comprises SK4A (SAT0037) or a derivative or salt thereof.

In some instances, a PDE inhibitor comprises a PDE inhibitor described in Chang, et al., “Imidazopyridine- and purine-thioacetamide derivatives: potent inhibitors of nucleotide pyrophosphatase/phosphodiesterase I (NPP1),”J. of Med. Chem., 57:10080-10100 (2014).

In some embodiments, a PDE inhibitor comprises a PDE inhibitor described in Lee, et al., “Thiazolo[3,2-a]benzimidazol-3(2H)-one derivatives: structure-activity relationships of selective nucleotide pyrophosphatase/phosphodiesterase1 (NPP1) inhibitors,” Bioorganic & Medicinal Chemistry, 24:3157-3165 (2016).

In some embodiments, a PDE inhibitor comprises a PDE inhibitor described in Shayhidin, et al., “Quinazoline-4-piperidine sulfamides are specific inhibitors of human NPP1 and prevent pathological mineralization of valve interstitial cells,” British Journal of Pharmacology, 172:4189-4199 (2015).

In some embodiments, a PDE inhibitor comprises a PDE inhibitor described in Li, et al., “Hydrolysis of 2′3′-cGAMP by ENPP-1 and design of nonhydrolyzable analogs,” Nature Chemical Biology, 10:1043-1048 (2014).

In some embodiments, a PDE inhibitor comprises Compound 1:

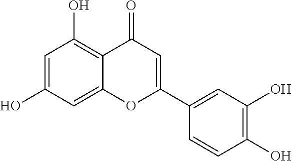 [SMILES: O=c1cc(-c2ccc(O)c(O)c2)oc2cc(O)cc(O)c12]

or a derivative, analog, or salt thereof.

In some embodiments, a PDE inhibitor comprises Compound 2:

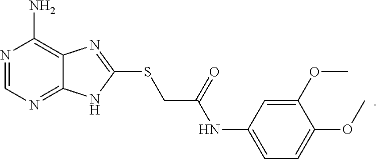 [SMILES: COc1ccc(NC(=O)CSc2nc3c(N)ncnc3[nH]2)cc1OC]

or a derivative, analog, or salt thereof.

In some embodiments, a PDE inhibitor comprises Compound 3:

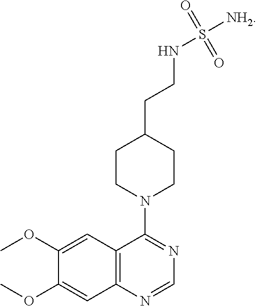 [SMILES: COc1cc2ncnc(N3CCC(CCNS(N)(=O)=O)CC3)c2cc1OC]

or a derivative, analog, or salt thereof.

In some embodiments, the infection is a viral infection, e.g., an infection from a DNA virus or a retrovirus. In some cases, the viral infection is due to herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In some instances, the infection is a bacterial infection, e.g., an infection from a Gram-negative bacterium or a Gram-positive bacterium. In some cases, the bacterium is Listeria monocytogenes, Mycobacterium tuberculosis, Francisella novicida, Legionella pneumophila, Chlamydia trachomatis, Streptococcus pneumoniae, or Neisseria gonorrhoeae.

In some cases, a PDE inhibitor is administered continuously for 1, 2, 3, 4, 5, 6, 7, 8, 9, 10, 14, 15, 28, 30 or more days. In some instances, the PDE inhibitor is administered continuously for 1 or more days. In some instances, the PDE inhibitor is administered continuously for 2 or more days. In some instances, the PDE inhibitor is administered continuously for 3 or more days. In some instances, the PDE inhibitor is administered continuously for 4 or more days. In some instances, the PDE inhibitor is administered continuously for 5 or more days. In some instances, the PDE inhibitor is administered continuously for 6 or more days. In some instances, the PDE inhibitor is administered continuously for 7 or more days. In some instances, the PDE inhibitor is administered continuously for 8 or more days. In some instances, the PDE inhibitor is administered continuously for 9 or more days. In some instances, the PDE inhibitor is administered continuously for 10 or more days. In some instances, the PDE inhibitor is administered continuously for 14 or more days. In some instances, the PDE inhibitor is administered continuously for 15 or more days. In some instances, the PDE inhibitor is administered continuously for 28 or more days. In some instances, the PDE inhibitor is administered continuously for 30 or more days.

In some cases, a PDE inhibitor is administered at predetermined time intervals for 1, 2, 3, 4, 5, 6, 7, 8, 9, 10, 14, 15, 28, 30 or more days. In some instances, the PDE inhibitor is administered at predetermined time intervals for 1 or more days. In some instances, the PDE inhibitor is administered at predetermined time intervals for 2 or more days. In some instances, the PDE inhibitor is administered at predetermined time intervals for 3 or more days. In some instances, the PDE inhibitor is administered at predetermined time intervals for 4 or more days. In some instances, the PDE inhibitor is administered at predetermined time intervals for 5 or more days. In some instances, the PDE inhibitor is administered at predetermined time intervals for 6 or more days. In some instances, the PDE inhibitor is administered at predetermined time intervals for 7 or more days. In some instances, the PDE inhibitor is administered at predetermined time intervals for 8 or more days. In some instances, the PDE inhibitor is administered at predetermined time intervals for 9 or more days. In some instances, the PDE inhibitor is administered at predetermined time intervals for 10 or more days. In some instances, the PDE inhibitor is administered at predetermined time intervals for 14 or more days. In some instances, the PDE inhibitor is administered at predetermined time intervals for 15 or more days. In some instances, the PDE inhibitor is administered at predetermined time intervals for 28 or more days. In some instances, the PDE inhibitor is administered at predetermined time intervals for 30 or more days.

In some embodiments, a PDE inhibitor is administered at predetermined time intervals for 1, 2, 3, 4, 5, 6, 7, 8, 9, 10, 11, 12, 24, 36 or more months. In some instances, the PDE inhibitor is administered at predetermined time intervals for 1 or more month. In some instances, the PDE inhibitor is administered at predetermined time intervals for 2 or more months. In some instances, the PDE inhibitor is administered at predetermined time intervals for 3 or more months. In some instances, the PDE inhibitor is administered at predetermined time intervals for 4 or more months. In some instances, the PDE inhibitor is administered at predetermined time intervals for 5 or more months. In some instances, the PDE inhibitor is administered at predetermined time intervals for 6 or more months. In some instances, the PDE inhibitor is administered at predetermined time intervals for 7 or more months. In some instances, the PDE inhibitor is administered at predetermined time intervals for 8 or more months. In some instances, the PDE inhibitor is administered at predetermined time intervals for 9 or more months. In some instances, the PDE inhibitor is administered at predetermined time intervals for 10 or more months. In some instances, the PDE inhibitor is administered at predetermined time intervals for 11 or more months. In some instances, the PDE inhibitor is administered at predetermined time intervals for 12 or more months. In some instances, the PDE inhibitor is administered at predetermined time intervals for 24 or more months. In some instances, the PDE inhibitor is administered at predetermined time intervals for 36 or more months.

In some cases, a PDE inhibitor is administered intermittently for 1, 2, 3, 4, 5, 6, 7, 8, 9, 10, 14, 15, 28, 30 or more days. In some instances, the PDE inhibitor is administered intermittently for 1 or more days. In some instances, the PDE inhibitor is administered intermittently for 2 or more days. In some instances, the PDE inhibitor is administered intermittently for 3 or more days. In some instances, the PDE inhibitor is administered intermittently for 4 or more days. In some instances, the PDE inhibitor is administered intermittently for 5 or more days. In some instances, the PDE inhibitor is administered intermittently for 6 or more days. In some instances, the PDE inhibitor is administered intermittently for 7 or more days. In some instances, the PDE inhibitor is administered intermittently for 8 or more days. In some instances, the PDE inhibitor is administered intermittently for 9 or more days. In some instances, the PDE inhibitor is administered intermittently for 10 or more days. In some instances, the PDE inhibitor is administered intermittently for 14 or more days. In some instances, the PDE inhibitor is administered intermittently for 15 or more days. In some instances, the PDE inhibitor is administered intermittently for 28 or more days. In some instances, the PDE inhibitor is administered intermittently for 30 or more days.

In some instances, a PDE inhibitor is administered to a subject at a therapeutically effective amount. For example, the therapeutically effective amount is optionally administered in 1 dose, 2 doses, 3 doses, 4 doses, 5 doses, 6 doses or more. In some instances, the therapeutically effective amount of a PDE inhibitor is administered to a subject in 1 dose. In some instances, the therapeutically effective amount of a PDE inhibitor is administered to a subject in 2 or more doses. In some instances, the therapeutically effective amount of a PDE inhibitor is administered to a subject in 3 or more doses. In some instances, the therapeutically effective amount of a PDE inhibitor is administered to a subject in 4 or more doses. In some instances, the therapeutically effective amount of a PDE inhibitor is administered to a subject in 5 or more doses. In some instances, the therapeutically effective amount of a PDE inhibitor is administered to a subject in 6 or more doses.

In some cases, the therapeutically effective amount of the PDE inhibitor selectively inhibits hydrolysis of 2′3′-cGAMP.

In some embodiments, the therapeutically effective amount of the PDE inhibitor further reduces ATP hydrolysis in PDE by less than 50%, less than 40%, less than 30%, less than 20%, less than 10%, less than 5%, or by less than 1% relative to the ATP hydrolysis of a PDE in the absence of the PDE inhibitor. In some cases, the therapeutically effective amount of the PDE inhibitor reduces ATP hydrolysis in PDE by less than 50% relative to the ATP hydrolysis of a PDE in the absence of the PDE inhibitor. In some cases, the therapeutically effective amount of the PDE inhibitor reduces ATP hydrolysis in PDE by less than 40% relative to the ATP hydrolysis of a PDE in the absence of the PDE inhibitor. In some cases, the therapeutically effective amount of the PDE inhibitor reduces ATP hydrolysis in PDE by less than 30% relative to the ATP hydrolysis of a PDE in the absence of the PDE inhibitor. In some cases, the therapeutically effective amount of the PDE inhibitor reduces ATP hydrolysis in PDE by less than 20% relative to the ATP hydrolysis of a PDE in the absence of the PDE inhibitor. In some cases, the therapeutically effective amount of the PDE inhibitor reduces ATP hydrolysis in PDE by less than 10% relative to the ATP hydrolysis of a PDE in the absence of the PDE inhibitor. In some cases, the therapeutically effective amount of the PDE inhibitor reduces ATP hydrolysis in PDE by less than 5% relative to the ATP hydrolysis of a PDE in the absence of the PDE inhibitor. In some cases, the therapeutically effective amount of the PDE inhibitor reduces ATP hydrolysis in PDE by less than 4% relative to the ATP hydrolysis of a PDE in the absence of the PDE inhibitor. In some cases, the therapeutically effective amount of the PDE inhibitor reduces ATP hydrolysis in PDE by less than 3% relative to the ATP hydrolysis of a PDE in the absence of the PDE inhibitor. In some cases, the therapeutically effective amount of the PDE inhibitor reduces ATP hydrolysis in PDE by less than 2% relative to the ATP hydrolysis of a PDE in the absence of the PDE inhibitor. In some cases, the therapeutically effective amount of the PDE inhibitor reduces ATP hydrolysis in PDE by less than 1% relative to the ATP hydrolysis of a PDE in the absence of the PDE inhibitor. In some cases, the therapeutically effective amount of the PDE inhibitor does not induce ATP hydrolysis in PDE.

In some embodiments, the subject is a human.

Method of Enhancing and/or Augmenting Type I IFN Production

In some embodiments, also described herein include a method of enhancing and/or augmenting type I interferon (IFN) production. In some instances, the method comprises an in vivo method. In some cases, the method comprises administering to a subject having an infection due to a pathogen a pharmaceutical composition comprising (i) an inhibitor of a 2′3′-cGAMP degradation polypeptide to block the hydrolysis of 2′3′-cGAMP; and (ii) a pharmaceutically acceptable excipient; wherein the presence of 2′3′-cGAMP activates the STING pathway, thereby enhancing the production of type I interferons.

In some cases, the 2′3′-cGAMP degradation polypeptide is a phosphodiesterase (PDE). In some cases, the 2′3′-cGAMP degradation polypeptide is a PDE5 protein. In some cases, the 2′3′-cGAMP degradation polypeptide is a PDE10 protein. In some cases, the 2′3′-cGAMP degradation polypeptide is a Pan-PDE protein. In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some embodiments, the inhibitor of a 2′3′-cGAMP degradation polypeptide is a PDE inhibitor. In some instances, the PDE inhibitor is a small molecule. In some instances, the PDE inhibitor is a PDE5 inhibitor. In some instances, the PDE inhibitor is a PDE10 inhibitor. In some instances, the PDE inhibitor is a Pan-PDE inhibitor. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some cases, the PDE inhibitor is a mixed inhibitor. In some embodiments, the PDE inhibitor binds to the catalytic domain of ENPP-1. In other embodiments, the PDE inhibitor binds to the nuclease-like domain of ENPP-1.

In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, the PDE inhibitor comprises 2-(3H-imidazo[4,5-b]pyridin-2-ylthio)-N-(3,4-dimethoxyphenyl)acetamide or a derivative, analog, or salt thereof.

In some instances, the PDE inhibitor comprises 2-(6-Amino-9H-purin-8-ylthio)-N-(3,4-dimethoxyphenyl)-acetamide, or a salt thereof.

In some cases, the PDE inhibitor comprises N-(3,4-Dimethoxyphenyl)-2-(5-methoxy-3H-imidazo[4,5-b]-pyridin-2-ylthio)acetamide or a salt thereof.

In some cases, the PDE inhibitor comprises 2-(1-(6,7-Dimethoxyquinazolin-4-yl)piperidin-4-yl)ethyl sulfamide or a salt thereof.

In some cases, the PDE inhibitor comprises ((1-(6,7-Dimethoxyquinazolin-4-yl)piperidin-4-yl)methyl)sulfamide or a salt thereof.

In some cases, the PDE inhibitor comprises SK4A (SAT0037) or a derivative or salt thereof.

In some embodiments, the PDE inhibitor comprises Compound 1:

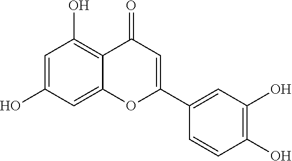 [SMILES: O=c1cc(-c2ccc(O)c(O)c2)oc2cc(O)cc(O)c12]

or a derivative, analog, or salt thereof.

In some embodiments, the PDE inhibitor comprises Compound 2:

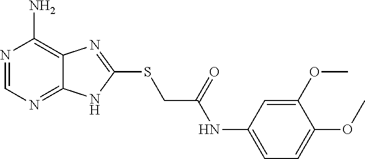 [SMILES: COc1ccc(NC(=O)CSc2nc3c(N)ncnc3[nH]2)cc1OC]

or a derivative, analog, or salt thereof.

In some embodiments, the PDE inhibitor comprises Compound 3:

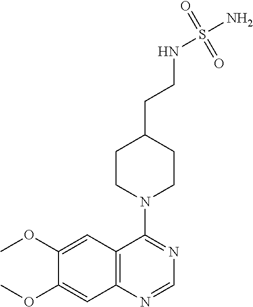 [SMILES: COc1cc2ncnc(N3CCC(CCNS(N)(=O)=O)CC3)c2cc1OC]

or a derivative, analog, or salt thereof.

In some embodiments, the infection is a viral infection, e.g., an infection from a DNA virus or a retrovirus. In some cases, the viral infection is due to herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In some instances, the infection is a bacterial infection, e.g., an infection from a Gram-negative bacterium or a Gram-positive bacterium. In some cases, the bacterium is Listeria monocytogenes, Mycobacterium tuberculosis, Francisella novicida, Legionella pneumophila, Chlamydia trachomatis, Streptococcus pneumoniae, or Neisseria gonorrhoeae.

In some cases, a PDE inhibitor is administered continuously for 1, 2, 3, 4, 5, 6, 7, 8, 9, 10, 14, 15, 28, 30 or more days. In some instances, the PDE inhibitor is administered continuously for 1 or more days. In some instances, the PDE inhibitor is administered continuously for 2 or more days. In some instances, the PDE inhibitor is administered continuously for 3 or more days. In some instances, the PDE inhibitor is administered continuously for 4 or more days. In some instances, the PDE inhibitor is administered continuously for 5 or more days. In some instances, the PDE inhibitor is administered continuously for 6 or more days. In some instances, the PDE inhibitor is administered continuously for 7 or more days. In some instances, the PDE inhibitor is administered continuously for 8 or more days. In some instances, the PDE inhibitor is administered continuously for 9 or more days. In some instances, the PDE inhibitor is administered continuously for 10 or more days. In some instances, the PDE inhibitor is administered continuously for 14 or more days. In some instances, the PDE inhibitor is administered continuously for 15 or more days. In some instances, the PDE inhibitor is administered continuously for 28 or more days. In some instances, the PDE inhibitor is administered continuously for 30 or more days.

In some cases, a PDE inhibitor is administered at predetermined time intervals for 1, 2, 3, 4, 5, 6, 7, 8, 9, 10, 14, 15, 28, 30 or more days. In some instances, the PDE inhibitor is administered at predetermined time intervals for 1 or more days. In some instances, the PDE inhibitor is administered at predetermined time intervals for 2 or more days. In some instances, the PDE inhibitor is administered at predetermined time intervals for 3 or more days. In some instances, the PDE inhibitor is administered at predetermined time intervals for 4 or more days. In some instances, the PDE inhibitor is administered at predetermined time intervals for 5 or more days. In some instances, the PDE inhibitor is administered at predetermined time intervals for 6 or more days. In some instances, the PDE inhibitor is administered at predetermined time intervals for 7 or more days. In some instances, the PDE inhibitor is administered at predetermined time intervals for 8 or more days. In some instances, the PDE inhibitor is administered at predetermined time intervals for 9 or more days. In some instances, the PDE inhibitor is administered at predetermined time intervals for 10 or more days. In some instances, the PDE inhibitor is administered at predetermined time intervals for 14 or more days. In some instances, the PDE inhibitor is administered at predetermined time intervals for 15 or more days. In some instances, the PDE inhibitor is administered at predetermined time intervals for 28 or more days. In some instances, the PDE inhibitor is administered at predetermined time intervals for 30 or more days.

In some embodiments, a PDE inhibitor is administered at predetermined time intervals for 1, 2, 3, 4, 5, 6, 7, 8, 9, 10, 11, 12, 24, 36 or more months. In some instances, the PDE inhibitor is administered at predetermined time intervals for 1 or more month. In some instances, the PDE inhibitor is administered at predetermined time intervals for 2 or more months. In some instances, the PDE inhibitor is administered at predetermined time intervals for 3 or more months. In some instances, the PDE inhibitor is administered at predetermined time intervals for 4 or more months. In some instances, the PDE inhibitor is administered at predetermined time intervals for 5 or more months. In some instances, the PDE inhibitor is administered at predetermined time intervals for 6 or more months. In some instances, the PDE inhibitor is administered at predetermined time intervals for 7 or more months. In some instances, the PDE inhibitor is administered at predetermined time intervals for 8 or more months. In some instances, the PDE inhibitor is administered at predetermined time intervals for 9 or more months. In some instances, the PDE inhibitor is administered at predetermined time intervals for 10 or more months. In some instances, the PDE inhibitor is administered at predetermined time intervals for 11 or more months. In some instances, the PDE inhibitor is administered at predetermined time intervals for 12 or more months. In some instances, the PDE inhibitor is administered at predetermined time intervals for 24 or more months. In some instances, the PDE inhibitor is administered at predetermined time intervals for 36 or more months.

In some cases, a PDE inhibitor is administered intermittently for 1, 2, 3, 4, 5, 6, 7, 8, 9, 10, 14, 15, 28, 30 or more days. In some instances, the PDE inhibitor is administered intermittently for 1 or more days. In some instances, the PDE inhibitor is administered intermittently for 2 or more days. In some instances, the PDE inhibitor is administered intermittently for 3 or more days. In some instances, the PDE inhibitor is administered intermittently for 4 or more days. In some instances, the PDE inhibitor is administered intermittently for 5 or more days. In some instances, the PDE inhibitor is administered intermittently for 6 or more days. In some instances, the PDE inhibitor is administered intermittently for 7 or more days. In some instances, the PDE inhibitor is administered intermittently for 8 or more days. In some instances, the PDE inhibitor is administered intermittently for 9 or more days. In some instances, the PDE inhibitor is administered intermittently for 10 or more days. In some instances, the PDE inhibitor is administered intermittently for 14 or more days. In some instances, the PDE inhibitor is administered intermittently for 15 or more days. In some instances, the PDE inhibitor is administered intermittently for 28 or more days. In some instances, the PDE inhibitor is administered intermittently for 30 or more days.

In some instances, a PDE inhibitor is administered to a subject at a therapeutically effective amount. For example, the therapeutically effective amount is optionally administered in 1 dose, 2 doses, 3 doses, 4 doses, 5 doses, 6 doses or more. In some instances, the therapeutically effective amount of a PDE inhibitor is administered to a subject in 1 dose. In some instances, the therapeutically effective amount of a PDE inhibitor is administered to a subject in 2 or more doses. In some instances, the therapeutically effective amount of a PDE inhibitor is administered to a subject in 3 or more doses. In some instances, the therapeutically effective amount of a PDE inhibitor is administered to a subject in 4 or more doses. In some instances, the therapeutically effective amount of a PDE inhibitor is administered to a subject in 5 or more doses. In some instances, the therapeutically effective amount of a PDE inhibitor is administered to a subject in 6 or more doses.

In some cases, the therapeutically effective amount of the PDE inhibitor selectively inhibits hydrolysis of 2′3′-cGAMP.

In some embodiments, the therapeutically effective amount of the PDE inhibitor further reduces ATP hydrolysis in PDE by less than 50%, less than 40%, less than 30%, less than 20%, less than 10%, less than 5%, or by less than 1% relative to the ATP hydrolysis of a PDE in the absence of the PDE inhibitor. In some cases, the therapeutically effective amount of the PDE inhibitor reduces ATP hydrolysis in PDE by less than 50% relative to the ATP hydrolysis of a PDE in the absence of the PDE inhibitor. In some cases, the therapeutically effective amount of the PDE inhibitor reduces ATP hydrolysis in PDE by less than 40% relative to the ATP hydrolysis of a PDE in the absence of the PDE inhibitor. In some cases, the therapeutically effective amount of the PDE inhibitor reduces ATP hydrolysis in PDE by less than 30% relative to the ATP hydrolysis of a PDE in the absence of the PDE inhibitor. In some cases, the therapeutically effective amount of the PDE inhibitor reduces ATP hydrolysis in PDE by less than 20% relative to the ATP hydrolysis of a PDE in the absence of the PDE inhibitor. In some cases, the therapeutically effective amount of the PDE inhibitor reduces ATP hydrolysis in PDE by less than 10% relative to the ATP hydrolysis of a PDE in the absence of the PDE inhibitor. In some cases, the therapeutically effective amount of the PDE inhibitor reduces ATP hydrolysis in PDE by less than 5% relative to the ATP hydrolysis of a PDE in the absence of the PDE inhibitor. In some cases, the therapeutically effective amount of the PDE inhibitor reduces ATP hydrolysis in PDE by less than 4% relative to the ATP hydrolysis of a PDE in the absence of the PDE inhibitor. In some cases, the therapeutically effective amount of the PDE inhibitor reduces ATP hydrolysis in PDE by less than 3% relative to the ATP hydrolysis of a PDE in the absence of the PDE inhibitor. In some cases, the therapeutically effective amount of the PDE inhibitor reduces ATP hydrolysis in PDE by less than 2% relative to the ATP hydrolysis of a PDE in the absence of the PDE inhibitor. In some cases, the therapeutically effective amount of the PDE inhibitor reduces ATP hydrolysis in PDE by less than 1% relative to the ATP hydrolysis of a PDE in the absence of the PDE inhibitor. In some cases, the therapeutically effective amount of the PDE inhibitor does not induce ATP hydrolysis in PDE.

In some embodiments, the subject is a human.

Methods of Inhibiting 2′3′-cGAMP Depletion

In some embodiments, further disclosed herein include methods of inhibiting depletion of 2′3′-cGAMP in a cell and selective inhibition of a 2′3′-cGAMP degradation polypeptide (e.g., ENPP-1). In some embodiments, disclosed herein includes a method of inhibiting depletion of 2′3′-cGAMP in a cell infected by a pathogen, which comprises contacting the cell infected by a pathogen and expressing a 2′3′-cGAMP degradation polypeptide with an inhibitor to generate a 2′3′-cGAMP degradation polypeptide-inhibitor adduct, thereby inhibiting the 2′3′-cGAMP degradation polypeptide from degrading 2′3′-cGAMP to prevent the depletion of 2′3′-cGAMP in the cell.

In some cases, the 2′3′-cGAMP degradation polypeptide is a phosphodiesterase (PDE). In some cases, the 2′3′-cGAMP degradation polypeptide is a PDE5 protein. In some cases, the 2′3′-cGAMP degradation polypeptide is a PDE10 protein. In some cases, the 2′3′-cGAMP degradation polypeptide is a Pan-PDE protein. In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some instances, disclosed herein includes a method of selectively inhibits a phosphodiesterase (PDE), which comprises contacting a cell characterized with an elevated population of cytosolic DNA with a PDE inhibitor to inhibit hydrolysis of 2′3-cGAMP, wherein the PDE inhibitor has a reduced function of ATP hydrolysis of the PDE, and wherein the elevated population of cytosolic DNA is generated by a virus. In some cases, the PDE inhibitor is a PDE5 inhibitor. In some cases, the PDE inhibitor is a PDE10 inhibitor. In some cases, the PDE inhibitor is a Pan-PDE inhibitor. In some cases, the PDE inhibitor is an ENPP-1 inhibitor.

In some instances, disclosed herein includes a method of selectively inhibits a phosphodiesterase (PDE), which comprises contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a virus.

In some instances, disclosed herein includes a method of selectively inhibits a phosphodiesterase (PDE), which comprises contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a virus.

In some embodiments, disclosed herein includes a method of selectively inhibits a phosphodiesterase (PDE), which comprises contacting a cell characterized with an elevated population of cytosolic DNA with a PDE inhibitor to inhibit hydrolysis of 2′3-cGAMP, wherein the PDE inhibitor has a reduced inhibition function ATP hydrolysis of the PDE, and wherein the elevated population of cytosolic DNA is generated by a recombinant DNA vaccine.

In some embodiments, disclosed herein includes a method of selectively inhibits a phosphodiesterase (PDE), which comprises contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a recombinant DNA vaccine.

In some embodiments, disclosed herein includes a method of selectively inhibits a phosphodiesterase (PDE), which comprises contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE, and wherein the elevated population of cytosolic DNA is generated by a recombinant DNA vaccine.

In some cases, the 2′3′-cGAMP degradation polypeptide is a phosphodiesterase (PDE). In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some instances, a method of selectively inhibits a phosphodiesterase (PDE) comprises contacting a cell characterized with an elevated population of cytosolic DNA with a PDE inhibitor to inhibit hydrolysis of 2′3-cGAMP, wherein the PDE inhibitor has a reduced inhibition function of ATP hydrolysis of the PDE. In some cases, the PDE inhibitor binds to the catalytic domain of ENPP-1. In some cases, the PDE inhibitor binds to the nuclease-like domain of ENPP-1.

In other instances, a method of selectively inhibits a phosphodiesterase (PDE) comprises contacting a cell characterized with an elevated population of cytosolic DNA with a catalytic domain-specific PDE inhibitor to inhibit hydrolysis of 2′3-cGAMP, wherein the PDE inhibitor has a reduced inhibition function of ATP hydrolysis of the PDE.

In additional instances, a method of selectively inhibits a phosphodiesterase (PDE) comprises contacting a cell characterized with an elevated population of cytosolic DNA with a nuclease-like domain-specific PDE inhibitor to inhibit hydrolysis of 2′3-cGAMP, wherein the PDE inhibitor has a reduced inhibition function of ATP hydrolysis of the PDE.

In some cases, the reduced inhibition function of ATP hydrolysis is relative to the ATP hydrolysis of a PDE in the absence of the PDE inhibitor. In some cases, the PDE inhibitor reduces ATP hydrolysis in the PDE by less than 50%, less than 40%, less than 30%, less than 20%, less than 10%, less than 5%, or to less than 1% relative to the ATP hydrolysis of a PDE in the absence of the PDE inhibitor. In some instances, the PDE inhibitor reduces ATP hydrolysis in the PDE by less than 50% relative to the ATP hydrolysis of a PDE in the absence of the PDE inhibitor. In some instances, the PDE inhibitor reduces ATP hydrolysis in the PDE by less than 40% relative to the ATP hydrolysis of a PDE in the absence of the PDE inhibitor. In some instances, the PDE inhibitor reduces ATP hydrolysis in the PDE by less than 30% relative to the ATP hydrolysis of a PDE in the absence of the PDE inhibitor. In some instances, the PDE inhibitor reduces ATP hydrolysis in the PDE by less than 20% relative to the ATP hydrolysis of a PDE in the absence of the PDE inhibitor. In some instances, the PDE inhibitor reduces ATP hydrolysis in the PDE by less than 10% relative to the ATP hydrolysis of a PDE in the absence of the PDE inhibitor. In some instances, the PDE inhibitor reduces ATP hydrolysis in the PDE by less than 5% relative to the ATP hydrolysis of a PDE in the absence of the PDE inhibitor. In some instances, the PDE inhibitor reduces ATP hydrolysis in the PDE by less than 4% relative to the ATP hydrolysis of a PDE in the absence of the PDE inhibitor. In some instances, the PDE inhibitor reduces ATP hydrolysis in the PDE by less than 3% relative to the ATP hydrolysis of a PDE in the absence of the PDE inhibitor. In some instances, the PDE inhibitor reduces ATP hydrolysis in the PDE by less than 2% relative to the ATP hydrolysis of a PDE in the absence of the PDE inhibitor. In some instances, the PDE inhibitor reduces ATP hydrolysis in the PDE by less than 1% relative to the ATP hydrolysis of a PDE in the absence of the PDE inhibitor. In some cases, the PDE inhibitor does not inhibit ATP hydrolysis of the PDE.

In some instances, the PDE inhibitor is a small molecule. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some cases, the PDE inhibitor is a mixed inhibitor. In some embodiments, the PDE inhibitor binds to the catalytic domain of ENPP-1. In other embodiments, the PDE inhibitor binds to the nuclease-like domain of ENPP-1.

In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, the PDE inhibitor comprises 2-(3H-imidazo[4,5-b]pyridin-2-ylthio)-N-(3,4-dimethoxyphenyl)acetamide or a derivative, analog, or salt thereof.

In some instances, the PDE inhibitor comprises 2-(6-Amino-9H-purin-8-ylthio)-N-(3,4-dimethoxyphenyl)-acetamide, or a salt thereof.

In some cases, the PDE inhibitor comprises N-(3,4-Dimethoxyphenyl)-2-(5-methoxy-3H-imidazo[4,5-b]-pyridin-2-ylthio)acetamide or a salt thereof.

In some cases, the PDE inhibitor comprises 2-(1-(6,7-Dimethoxyquinazolin-4-yl)piperidin-4-yl)ethyl sulfamide or a salt thereof.

In some cases, the PDE inhibitor comprises ((1-(6,7-Dimethoxyquinazolin-4-yl)piperidin-4-yl)methyl)sulfamide or a salt thereof.

In some cases, the PDE inhibitor comprises SK4A (SAT0037) or a derivative or salt thereof.

In some embodiments, the PDE inhibitor comprises Compound 1:

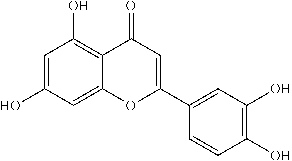 [SMILES: O=c1cc(-c2ccc(O)c(O)c2)oc2cc(O)cc(O)c12]

or a derivative, analog, or salt thereof.

In some embodiments, the PDE inhibitor comprises Compound 2:

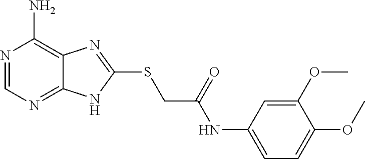 [SMILES: COc1ccc(NC(=O)CSc2nc3c(N)ncnc3[nH]2)cc1OC]

or a derivative, analog, or salt thereof.

In some embodiments, the PDE inhibitor comprises Compound 3:

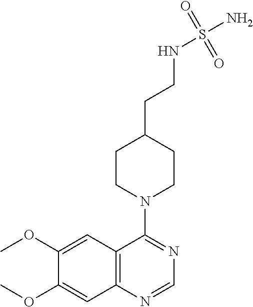 [SMILES: COc1cc2ncnc(N3CCC(CCNS(N)(=O)=O)CC3)c2cc1OC]

or a derivative, analog, or salt thereof.

In some embodiments, the infection is a viral infection, e.g., an infection from a DNA virus or a retrovirus. In some cases, the viral infection is due to herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In some instances, the infection is a bacterial infection, e.g., an infection from a Gram-negative bacterium or a Gram-positive bacterium. In some cases, the bacterium is Listeria monocytogenes, Mycobacterium tuberculosis, Francisella novicida, Legionella pneumophila, Chlamydia trachomatis, Streptococcus pneumoniae, or Neisseria gonorrhoeae.

In some instances, the cytosolic DNA comprises viral DNA. In some cases, the elevated population of cytosolic DNA is due to a viral infection to the host cell. In other cases, the elevated population of the cytosolic DNA is due to delivery of viral DNA through a virus-like particle (VLP).

In some instances, the elevated population of cytosolic DNA is due to a recombinant DNA vaccine, which comprises a DNA vector encoding a viral antigen. In some cases, the viral antigen is derived from a DNA virus. In other cases, the viral antigen is derived from a retrovirus. In some cases, the viral antigen is derived from herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In some cases, the recombinant DNA vaccine comprise a DNA vector that encodes a bacterial antigen, e.g., derived from a Gram-negative bacterium or a Gram-positive bacterium. In some cases, the bacterial antigen is derived from Listeria monocytogenes, Mycobacterium tuberculosis, Francisella novicida, Legionella pneumophila, Chlamydia trachomatis, Streptococcus pneumoniae, or Neisseria gonorrhoeae.

In some embodiments, a DNA vector described herein comprises a circular plasmid or a linear nucleic acid. In some cases, the circular plasmid or linear nucleic acid is capable of directing expression of a particular nucleotide sequence in an appropriate subject cell. In some cases, the vector has a promoter operably linked to the microbial antigen-encoding nucleotide sequence, which is operably linked to termination signals. In some instances, the vector also contains sequences required for proper translation of the nucleotide sequence. The vector comprising the nucleotide sequence of interest can be chimeric, meaning that at least one of its components is heterologous with respect to at least one of its other components. The expression of the nucleotide sequence in the expression cassette can be under the control of a constitutive promoter or of an inducible promoter, which can initiate transcription only when the host cell is exposed to some particular external stimulus.

In some instances, the vector is a plasmid. In some cases, the plasmid is useful for transfecting cells with nucleic acid encoding the microbial antigen, which the transformed host cells can be cultured and maintained under conditions wherein production of the microbial antigen takes place.

In some instances, the plasmid comprises a mammalian origin of replication in order to maintain the plasmid extrachromosomally and produce multiple copies of the plasmid in a cell. The plasmid can be pVAXI, pCEP4 or pREP4 from Invitrogen (San Diego, Calif.).

In some instances, the plasmid further comprises a regulatory sequence, which enables gene expression in a cell into which the plasmid is administered. In some cases, the coding sequence further comprises a codon that allows for more efficient transcription of the coding sequence in the host cell.

In some instances, the vector is a circular plasmid, which transforms a target cell by integration into the cellular genome or exist extrachromosomally (e.g., autonomous replicating plasmid with an origin of replication). Exemplary vectors include pVAX, pcDNA3.0, or provax, or any other expression vector capable of expressing DNA encoding the antigen and enabling a cell to translate the sequence to an antigen that is recognized by the immune system.

In some instances, the recombinant nucleic acid vaccine comprises a viral vector. Exemplary viral based vectors include adenoviral based, lentivirus based, adeno-associated (AAV) based, retroviral based, or poxvirus based vectors.

In some instances, the recombinant DNA vaccine is a linear DNA vaccine, or linear expression cassette (“LEC”), that is capable of being efficiently delivered to a subject via electroporation and expressing one or more polypeptides disclosed herein. The LEC can be any linear DNA devoid of any phosphate backbone. The DNA can encode one or more microbial antigens. The LEC can contain a promoter, an intron, a stop codon, and/or a polyadenylation signal. In some cases, the LEC does not contain any antibiotic resistance genes and/or a phosphate backbone. In some cases, the LEC does not contain other nucleic acid sequences unrelated to the microbial antigen.

### Method of Activating a STING Protein Dimer

In some embodiments, a method of stabilizing a stimulator of interferon genes (STING) protein dimer in a cell, which comprises (a) contacting the cell infected by a pathogen and characterized with an elevated population of cytosolic DNA with an inhibitor of a 2′3′-cGAMP degradation polypeptide to inhibit hydrolysis of 2′3′-cGAMP; and (b) interacting 2′3′-cGAMP to a STING protein dimer to generate a 2′3′-cGAMP-STING complex, thereby stabilizing the STING protein dimer.

In some cases, the 2′3′-cGAMP degradation polypeptide is a phosphodiesterase (PDE). In some cases, the 2′3′-cGAMP degradation polypeptide is a PDE5 protein. In some cases, the 2′3′-cGAMP degradation polypeptide is a PDE10 protein. In some cases, the 2′3′-cGAMP degradation polypeptide is a Pan-PDE protein. In some cases, the 2′3′-cGAMP degradation polypeptide is an ectonucleotide pyrophosphatase/phosphodiesterase (ENPP) protein. In some cases, the 2′3′-cGAMP degradation polypeptide is ectonucleotide pyrophosphatase/phosphodiesterase family member 1 (ENPP-1).

In some instances, the PDE inhibitor is a small molecule. In some instances, the PDE inhibitor is a PDE5 inhibitor. In some instances, the PDE inhibitor is a PDE10 inhibitor. In some instances, the PDE inhibitor is a Pan-PDE inhibitor. In some instances, the PDE inhibitor is an ENPP-1 inhibitor. In some cases, the PDE inhibitor is a reversible inhibitor. In some cases, the PDE inhibitor is a competitive inhibitor. In some cases, the PDE inhibitor is an allosteric inhibitor. In other cases, the PDE inhibitor is an irreversible inhibitor. In some cases, the PDE inhibitor is a mixed inhibitor. In some embodiments, the PDE inhibitor binds to the catalytic domain of ENPP-1. In other embodiments, the PDE inhibitor binds to the nuclease-like domain of ENPP-1.

In some embodiments, the PDE inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative or PSB-POM141.

In some instances, the PDE inhibitor comprises 2-(3H-imidazo[4,5-b]pyridin-2-ylthio)-N-(3,4-dimethoxyphenyl)acetamide or a derivative, analog, or salt thereof.

In some instances, the PDE inhibitor comprises 2-(6-Amino-9H-purin-8-ylthio)-N-(3,4-dimethoxyphenyl)-acetamide, or a salt thereof.

In some cases, the PDE inhibitor comprises N-(3,4-Dimethoxyphenyl)-2-(5-methoxy-3H-imidazo[4,5-b]-pyridin-2-ylthio)acetamide or a salt thereof.

In some cases, the PDE inhibitor comprises 2-(1-(6,7-Dimethoxyquinazolin-4-yl)piperidin-4-yl)ethyl sulfamide or a salt thereof.

In some cases, the PDE inhibitor comprises ((1-(6,7-Dimethoxyquinazolin-4-yl)piperidin-4-yl)methyl)sulfamide or a salt thereof.

In some cases, the PDE inhibitor comprises SK4A (SAT0037) or a derivative or salt thereof.

In some embodiments, the PDE inhibitor comprises Compound 1:

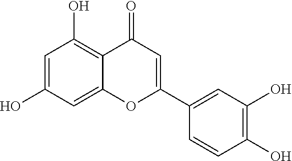 [SMILES: O=c1cc(-c2ccc(O)c(O)c2)oc2cc(O)cc(O)c12]

or a derivative, analog, or salt thereof.

In some embodiments, the PDE inhibitor comprises Compound 2:

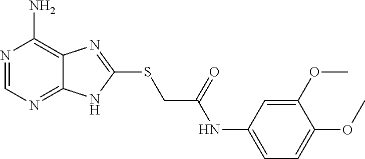 [SMILES: COc1ccc(NC(=O)CSc2nc3c(N)ncnc3[nH]2)cc1OC]

or a derivative, analog, or salt thereof.

In some embodiments, the PDE inhibitor comprises Compound 3:

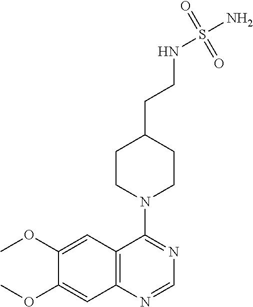 [SMILES: COc1cc2ncnc(N3CCC(CCNS(N)(=O)=O)CC3)c2cc1OC]

or a derivative, analog, or salt thereof.

As described above, the pathogen is optionally a virus or a bacterium. In some cases, the pathogen is a virus, e.g., a DNA virus or a retrovirus. In some cases, the pathogen comprises herpes simplex virus 1 (HSV-1), murine gamma-herpesvirus 68 (MHV68), Kaposi's sarcoma-associated herpesvirus (KSHV), vaccinia virus (VACV), adenovirus, human papillomaviruses (HPV), hepatitis B virus (HBV), human immunodeficiency virus (HIV), or human cytomegalovirus (HCMV).

In other instances, the pathogen is a bacterium, e.g., a Gram-negative bacterium or a Gram-positive bacterium. In some cases, the pathogen comprises Listeria monocytogenes, Mycobacterium tuberculosis, Francisella novicida, Legionella pneumophila, Chlamydia trachomatis, Streptococcus pneumoniae, or Neisseria gonorrhoeae.

### Additional Therapeutic Agents

In some embodiments, one or more methods described herein further comprising administering an additional therapeutic agent. In some instances, the additional therapeutic agent comprises an antimicrobial agent. An antimicrobial is an agent that kills microorganisms or inhibits their growth. In some instances, antimicrobial agents are grouped according to the microorganisms they act primarily against. For example, antibiotics are used against bacteria. In some cases, antimicrobials are also classified according to their function. Agents that kill microbes are called microbicidal, while those that merely inhibit their growth are called biostatic. The use of antimicrobial agents to treat infection is known as antimicrobial chemotherapy, while the use of antimicrobial medicines to prevent infection is known as antimicrobial prophylaxis.

In some embodiments, the classes of antimicrobial agents are are further classified into antibiotics and antivirals. The term “antibiotic” includes formulations derived from living organisms as well as synthetic antimicrobials.

In some embodiments, an additional therapeutic agent described herein is an antibacterial agent. Antibacterials are used to treat bacterial infections. Antibacterial agents are further subdivided into bactericidal agents, which kill bacteria, and bacteriostatic agents, which slow down or stall bacterial growth.

In some embodiments, an additional therapeutic agent described herein is an antiviral agent. Antiviral agents are a class of medication used specifically for treating viral infections. Like antibiotics, specific antivirals are used for specific viruses. Many of the antiviral drugs available are designed to treat infections by retroviruses, e.g., HIV. In some instances, an exemplary class of antiretroviral drugs includes the class of protease inhibitors.

In some embodiments, an inhibitor of a 2′3′-cGAMP degradation polypeptide and an additional therapeutic agent are administered simultaneously. In other instances, an inhibitor of a 2′3′-cGAMP degradation polypeptide and an additional therapeutic agent are administered sequentially. In some cases, an inhibitor of a 2′3′-cGAMP degradation polypeptide is administered before administering an additional therapeutic agent. In other cases, an inhibitor of a 2′3′-cGAMP degradation polypeptide is administered after administering an additional therapeutic agent.

### Pharmaceutical Compositions and Formulations

In certain embodiments, disclosed herein include pharmaceutical compositions and formulations comprising a compound described herein. In some embodiments, the pharmaceutical compositions described herein are formulated for administering to a subject by systemic administration. In other embodiments, the pharmaceutical compositions described herein are formulated for administering to a subject by local administration. In some instances, the administration routes include, but are not limited to, parenteral (e.g., intravenous, subcutaneous, intramuscular, intracerebral, intracerebroventricular, intra-articular, intraperitoneal, or intracranial), oral, sublingual, intranasal, buccal, rectal, or transdermal administration routes. In some instances, the pharmaceutical composition describe herein is formulated for parenteral (e.g., intravenous, subcutaneous, intramuscular, intracerebral, intracerebroventricular, intra-articular, intraperitoneal, or intracranial) administration. In other instances, the pharmaceutical composition describe herein is formulated for oral administration. In additional instances, the pharmaceutical composition describe herein is formulated for sublingual administration. In additional instances, the pharmaceutical composition describe herein is formulated for intranasal administration. In some cases, the pharmaceutical composition is administered to a subject as an injection. In other instances, the pharmaceutical composition is administered to a subject as an infusion.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens for a Pharmaceutical Composition

In some embodiments, a pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a PDE inhibitor, optionally with one or more additional therapeutic agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, e.g., ±5%, ±10%, or ±15%.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. For example the term “treat” or “treating” with respect to a microbial infection refers to stopping the progression of said infection, slowing down, or amelioration of symptoms associated with the presence of said cells. Treatment of an individual suffering from an infectious disease organism refers to a decrease and elimination of the disease organism from an individual. For example, a decrease of viral particles as measured by plaque forming units or other automated diagnostic methods such as ELISA etc.

By “therapeutically effective amount” is meant an amount of a compound described herein effective to yield the desired therapeutic response. For example, an amount effective to delay the spread of infection or to eliminate the source of infection. The therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, “derivative” refers to a chemically or biologically modified version of a chemical compound that is structurally similar to a parent compound and (actually or theoretically) derivable from that parent compound. In some cases, a derivative has different chemical or physical properties relative to the parent compound. For example, the derivative may be more hydrophilic or it may have altered reactivity as compared to the parent compound. Derivatization (i.e., modification) may involve substitution of one or more moieties within the molecule (e.g., a change in functional group) that do not substantially alter the function of the molecule for a desired purpose. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analogue” refers to a chemical compound that is structurally similar to another but differs slightly in composition (as in the replacement of one atom by an atom of a different element or in the presence of a particular functional group), but may or may not be derivable from the parent compound. A “derivative” differs from an “analogue” in that a parent compound may be the starting material to generate a “derivative,” whereas the parent compound may not necessarily be used as the starting material to generate an “analogue.”

### EXAMPLES

These examples are provided for illustrative purposes only and not to limit the scope of the claims provided herein.

### Example 1. ATP Hydrolysis

ENPP-1 is an ectonucleotidase which hydrolyze the STING substrate 2′,3′-cGAMP. In some instances, an inhibitor of ENPP-1 is capable of selectively block the hydrolysis of 2′,3′-cGAMP but reduces or minimally inhibits the hydrolysis of ATP. In some cases, an ATP hydrolysis assay is used to measure the selectively of a ENPP-1 inhibitor. The following table 1 provides illustrative ENPP-1 inhibitors to be used with this experiment.

| ENPP-1 Inhibitor | Conc. 1 | Conc. 2 | Conc. 3 |
|---|---|---|---|
| ARL67156 | 10 μM | 100 μM | 1 mM |
| adenosine 5′-(α-borano)- | 10 μM | 100 μM | 1 mM |
| β,γ-methylene triphosphate | | | |
| N-(3,4-Dimethoxyphenyl)-2-(5-methoxy-3H- | 10 μM | 100 μM | 1 mM |
| imidazo[4,5-b]-pyridin-2-ylthio)acetamide | | | |

A 50 μL solution comprising 50 mM Tris-HCl, 200 mM NaCl, 0.1 mM CaCl₂, 1 ng/4 purified ENPP-1, and optionally with a ENPP-1 inhibitor, at pH 7.6, is prepared. The reaction is initiated with the addition of AMP-pNP and is incubated for about 10 minutes at a temperature of about 37° C. The rate of product release is monitored continuously by measuring the OD at 405 nm. The specific activity is calculated as follows:

Specific activity (pmol/min/μg)=[Adjusted V max*(OD/min)*Conversion Factor #(pmo/OD)]/μg enzyme

# Conversion Factor is derived using calibration standard 4-Nitrophenol.

A control is prepared to establish background signal.

### Example 2. Indirect Quantitation of 2′,3′-cGAMP Hydrolysis

Hydrolysis of 2′,3′-cGMP generates GMP which subsequently releases free phosphate in the presence of a phosphatase. In some instances, the production of free phosphate is used to measure the selectivity of a ENPP-1 inhibitor.

A 50 μL solution comprising 50 mM Tris-HCl, 200 mM NaCl, 0.1 mM CaCl₂, 1 ng/4 purified ENPP-1, and optionally with a ENPP-1 inhibitor, at pH 7.6, is prepared. The reaction is initiated with the addition of 2′3′-cGAMP and is incubated for about 10 minutes at a temperature of about 37° C. The assay is stopped by adding a cocktail of MgCl₂, a chelator, an alkaline phosphatase and a ENPP-1 inhibitor. The rate of free phosphate is detected using a Malachite Green Phosphate Detection kit. The following table 2 provides illustrative ENPP-1 inhibitors to be used with this experiment.

| ENPP-1 Inhibitor | Conc. 1 | Conc. 2 | Conc. 3 |
|---|---|---|---|
| ARL67156 | 10 μM | 100 μM | 1 mM |
| adenosine 5′-(α-borano)-β,γ-methylene | 10 μM | 100 μM | 1 mM |
| triphosphate | | | |
| N-(3,4-Dimethoxyphenyl)-2-(5-methoxy-3H- | 10 μM | 100 μM | 1 mM |
| imidazo[4,5-b]-pyridin-2-ylthio)acetamide | | | |

The specific activity is calculated as follows:

Specific activity (pmol/min/μg)=[Adjusted V max*(OD/min)*Conversion Factor(pmo/OD)]/μg enzyme

### Example 3. In Silico Design of ENPP-1 Inhibitors

Ligand-based virtual screening is carried out with a known ENPP-1 inhibitor using the Schrödinger/E-pharmacophore modeling software. A 2D similarity search is conducted using Radial-ECFP-DL2 and MOLPRINT2D methods. An initial hit is set at 10,000 with subsequent refinements based on the number and strength of ligand-site residue interactions.

ENPP-1 PDB structures that are used during the in silico screening include 4GTW, 4GTX, 4GTY and 4GTZ.

### Example 4. Measuring ATP Hydrolysis by ENPP1

ENPP1 is an ectonucleotidase that hydrolyzes both the STING activator 2′,3′-cGAMP and 5′ATP (ATP). In some instances, an inhibitor of ENPP-1 is capable of selectively blocking the hydrolysis of 2′,3′-cGAMP while only minimally inhibiting the hydrolysis of ATP. The ATP analog p-Nitrophenyl 5′-Adenosine Monophosphate (AMP-pNP) has been demonstrated to accurately reflect hydrolysis of native ATP by different classes of ENPP1 inhibitors₁ and was synthesized as described before (Lee at al. Substrate-Dependence of Competitive Nucleotide Pyrophosphatase/Phosphodiesterase1 (NPP1) Inhibitors. Front Pharmacol. 2017 Feb. 15; 8:54). The ENPP1 assay with AMP-pNP substrate is conducted in a buffer containing 50 mM Tris-HCl (pH 8.5)/250 mM NaCl/0.5 mM CaCl₂/1 μM ZnCl₂/0.1% DMSO. Inhibitors are added at final concentrations ranging between 10 μM and 30 pM depending on the compound. Duplicate wells are run at each inhibitor concentration. The final assay volume is 40 μL and human recombinant ENPP1 is present at 60 ng/well. The assay is initiated by the addition of substrate (300 μM AMP-pNP final concentration), and incubated for 20 minutes at 37° C. The absorbance at 405 nm is then read in a Tecan® plate reader. Each assay plate also includes wells with no enzyme added (MIN OD) and wells with no inhibitor added (MAX OD). The percent inhibition of ENPP1 for each sample is then calculated as:

% inhibition={[Average of (MAX OD−MIN OD)−(sample OD−Average MIN OD)]/Average of (MAX OD−MIN OD)}×100%.

IC₅₀ values of compounds were calculated by entering the percent inhibition values into a sigmoidal variable slope nonlinear regression model in GraphPad Prism® software. IC50 values were converted to Ki values using the Cheng-Prusoff equation, where the Kₘ was 151 μM, based on internal determinations

$${K}_{i} = \frac{{IC}_{50}}{1 + \frac{[ S ]}{{K}_{m}}}$$

### Example 5. Quantitation of 2′,3′-cGAMP Hydrolysis

Hydrolysis of 2′,3′-cGAMP by ENPP1 generates the products 5′-GMP and 5′-AMP. In some instances, ENPP1 activity with 2′,3′-cGAMP substrate is measured using the AMP-Glo™ Assay kit (Promega Corporation) to quantitate the production of 5′-AMP. The AMP-Glo™ Assay Kit contains two reagents that are added sequentially. The first converts the 5′-AMP produced in the reaction to 5′ADP. The second converts the 5′-ADP to 5′ATP and reacts the 5′-ATP with the luciferase/luciferin pair to produce the luminescence signal. The ENPP1 assay with 2′,3′-cGAMP substrate is conducted in a buffer containing 50 mM Tris-HCl (pH 8.5)/250 mM NaCl/0.5 mM CaCl₂/1 μM ZnCl₂/0.1% DMSO. Inhibitors are added at final concentrations ranging between 10 μM and 30 pM depending on the compound. Duplicate wells are run at each inhibitor concentration. The final assay volume is 18 μL and human recombinant ENPP1 is present at 5 ng/well. The assay is initiated by the addition of substrate (20 μM 2′3′cGAMP final concentration), and incubated for 30 minutes at 37° C. To stop the reaction, 12 μl of AMP-Glo reagent I is added and the plate is incubated for 60 minutes at room temperature. 25 μl of AMP-detection reagent is then added and the wells are again incubated for 60 minutes at room temperature. The luminescence signal is then measured using a plate-reading luminometer. Each assay plate also includes wells with no enzyme added (MIN OD) and wells with no inhibitor added (MAX OD). The percent inhibition of ENPP1 for each sample is then calculated as:

% inhibition={[Average of (MAX OD−MIN OD)−(sample OD−Average MIN OD)]/Average of (MAX OD−MIN OD)}×100%.

IC50 values of compounds were calculated by entering the percent inhibition values into a sigmoidal variable slope nonlinear regression model in GraphPad Prism® software. IC50 values were converted to Ki values using the Cheng-Prusoff equation₂ where the Kₘ was 15 μM, based on internal determinations.

$${K}_{i} = \frac{{IC}_{50}}{1 + \frac{[ S ]}{{K}_{m}}}$$

### Example 6. PDE Inhibitors Library Screen on cGAMP Activated THP-1 Cells

Materials:

2′,3′-cGAMP (InvivoGen, catalog # tlrl-nacga23)—A STING agonist sensitive to hydrolysis by ENPP-1.

2′,3′-cGAM(PS)2 (Rp/Sp) (InvivoGen, cat # tlrl-nacga2srs)—A STING agonist resistant to hydrolysis by ENPP-1 (measure of maximum IFNβ response in absence of ENPP-1 degradation of STING agonist).

IFNβ Assay Kit: VeriKine Human Interferon Beta ELISA Kit (PBL Assay Science, catalog #41410). Standard range in the kit (pg/mL): 50, 100, 200, 400, 1000, 2000, 4000.

Controls:

Negative control: unstimulated THP-1 cells (no 2′,3′-cGAMP or 2′,3′-cGAMP(PS)2(Rp/Sp)).

Vehicle control: 0.1% DMSO (control where no compounds were added—vehicle used to dissolve compounds. 10 uL media (control used for wells where 2′,3′-cGAMP or 2′,3′-cGAMP(PS)2(Rp/Sp) was not added))

Positive controls: 2′,3′-cGAMP sensitive to ENPP1 hydrolysis and 2′,3′-cGAM(PS)2 (Rp/Sp) insensitive to ENPP1 hydrolysis

IFNβ Sample Analysis Dilution Factor:

Neat samples (no dilution) analyzed for IFNβ in samples of 50 μL.

THP-1 Cell Activation and Screening of Test Compounds:
(a) THP-1 cells from bulk cultures were counted and suspended in RPMI 1640, 20% FBS, 2.5 mM L-alanyl-L-glutamine at a concentration of 5.5×10⁶ cells/mL. The THP-1 cells were subsequently seeded into 96 well round bottom plates—volume of 180 μL/well (1×10⁶ cells per well), and the plate was incubated for 1 hour at 37° C., 5% CO₂.
(b) Test compounds comprising known phosphodiesterase inhibitors (PDEs), were screened in triplicate (N1, N2, N3) on THP-1 cells. For compound screening, test compounds were assayed at a final concentration of 10 μM. A 200 μM (0.2 mM) working solution of each compound was prepared by diluting 10 mM DMSO stock solutions of each compound in media at a dilution of 50:1 (1640, 20% FBS, 2.5 mM L-alanyl-L-glutamine). Working solutions of each test compound were added to triplicate wells containing 180 uL of THP-1 cell suspension, in a volume of 10 μL to achieve a 10 μM concentration following the addition of a 10 μL volume 2′,3′-cGAMP in the final step to activate the STING pathway.
(c) The STING agonist 2′,3′-cGAMP ligand provided as a sterile powder by InvivoGen was diluted in sterile, endotoxin-free water, as per the manufacturer's instructions to yield a 1 mg/mL solution (1.4 mM or 1400 μM). Working solutions of 2′,3′-cGAMP at concentrations of 600 and 800 or 1000 μM (20× the final concentrations used to activate the STING pathway in the THP-1 cells) were prepared by making the small dilutions of the 1 mg/mL stock of 2′,3′-cGAMP with the cell culture media.
(d) Following a 1-hour pre-incubation of the cells with the various test compounds, the STING pathway was activated by the addition of 2′,3′-cGAMP. Final concentrations of 2′,3′-cGAMP used to activate the THP-1 cells were 30 μM and a higher concentration of 40 or 50 μM. Vehicle control wells where 2′,3′-cGAMP ligand is added at concentrations of 30 and 50 μM in the absence of any test compounds were included to assess baseline activation of the THP-1 cells.
(e) The nonhydrolyzable form of 2′,3′-cGAMP or 2′,3′-cGAM(PS)2 (Rp/Sp) was included as an additional positive control—tested at concentrations of 40, and 80 μM in duplicate or triplicate. This agonist represents maximum activation of the STING pathway in the absence of the agonist degradation as occurs with native 2′,3′-cGAMP, with an IFNβ response higher than the response seen with native 2′,3′-cGAMP.
(f) “Vehicle only” control wells in triplicate were included on each plate to assess the basal level of STING activation in the absence of agonist.
(g) Final assay volume were 200 μL/well
   1) THP-1 cells=180 μL
   2) test compounds=10 μL
   3) 2′,3′-cGAMP or 2′,3′-cGAM(PS)2 (Rp/Sp) or vehicle control=10 μL
(h) Plates were incubated for 24 hours at 37° C., 5% CO₂.

(i) Cell culture supernatants were harvested by centrifuging the plates at 200×g for 10 minutes. Cell culture supernatants were then transferred to a clean plate and stored at −80° C. until ready to analyze for IFNβ levels.
(j) IFNβ levels in cell culture supernatants were determined by ELISA according to the manufacturer's instructions (VeriKine Human IFNβ Assay).
(k) The interpolated data was normalized to vehicle control, or unstimulated control, and analyzed.

Results:

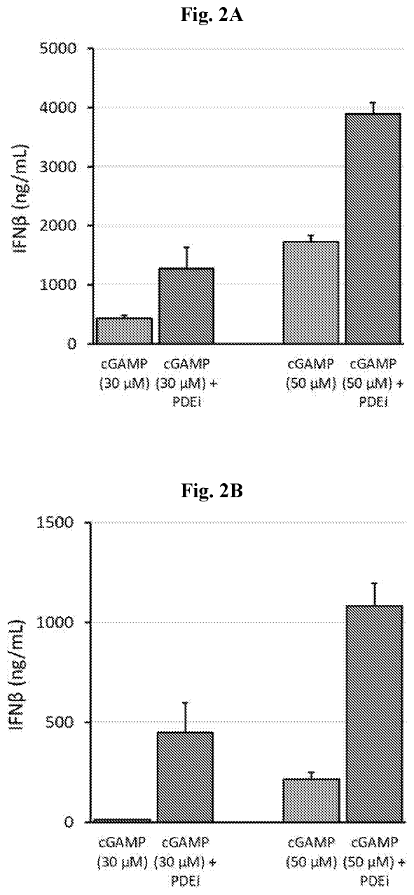

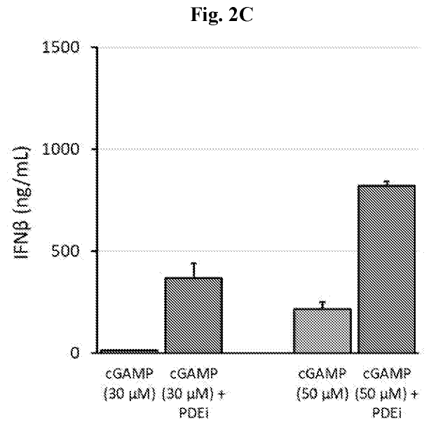

FIG. 2A-FIG. 2C illustrate exemplary compounds identified in the screen that augment cGAMP mediated IFNβ production.

Compounds found to augment IFNβ production in THP-1 cells activated with a suboptimal concentration of 2′,3′-cGAMP were evaluated for inhibition of ENPP1 mediated hydrolysis of 2′3′-cGAMP, as described in Example 5 using the AMP-GLO method. The following table 3 exemplifies the compounds that are inhibitors of ENPP1. Inhibition of 2′3′-cGAMP hydrolysis by ENPP1 in the presence of Compounds 1-3 at concentrations of 1 and 10 μM is shown.

| | Inhibition of ENPP1 with |
|---|---|
| Compound | cGAMP as the Substrate |
| 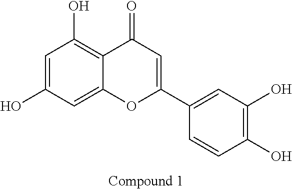 [SMILES: O=c1cc(-c2ccc(O)c(O)c2)oc2cc(O)cc(O)c12] | 99% Inhibition at 10 μM 76% Inhibition at 1 μM |
| 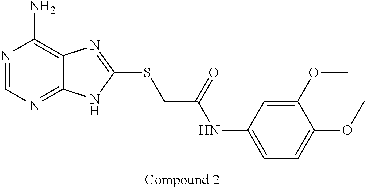 [SMILES: COc1ccc(NC(=O)CSc2nc3c(N)ncnc3[nH]2)cc1OC] | 99% Inhibition at 10 μM 99% Inhibition at 1 μM |
| 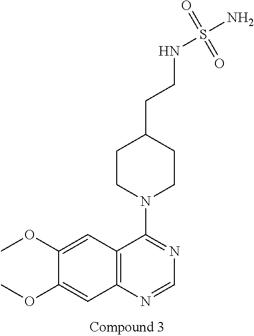 [SMILES: COc1cc2ncnc(N3CCC(CCNS(N)(=O)=O)CC3)c2cc1OC] | 99% Inhibition at 10 μM 99% Inhibition at 1 μM |

Inhibitor Selectivity Table

ENPP-1 catalyzes the hydrolysis of both 2′3′-cGAMP and ATP substrates. Compounds were tested for inhibition of ENPP-1 mediated hydrolysis of both the 2′3′-cGAMP and AMP-pNP (an analog of ATP) substrate to assess compound selectivity using methods described in Examples 4 and 5. In table 4 below, the potency of compounds to inhibit 2′3′cGAMP and AMP-pNP substrate hydrolysis by ENPP1 is provided as Ki determinations (nM). Additionally, the selectivity ratio for 2′3′c-GAMP versus AMP-pNP substrate inhibition has been calculated [Ki (AMP-pNP)/Ki (2′3′-cGAMP)]. The selectivity for inhibition of cGAMP over AMP-pNP hydrolysis by ENPP1 ranges from ˜6.8-fold for Compound 25, up to >37,500-fold for Compound 4. These results demonstrate that it is possible to identify inhibitors of ENPP-1 that selectively block the hydrolysis of 2′3′-cGAMP, while having a limited effect on the hydrolysis of the ATP analog.

| | | | | | Substrate |
|---|---|---|---|---|---|
| | | | ENPP1 inhibition | | Selectivity |
| | | | Ki (nM) | | Ki Ratio |
| | | | cGAMP | AMP-pNP | AMP- |
| Compound | Structure | Series | Substrate | Substrate | pNP/2′3′cGAMP |
| Compound 2 | 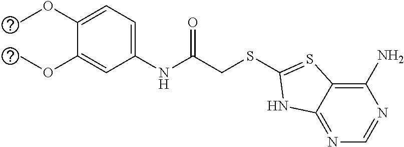 | 2 | 1.2 | >3000 | >2500 |
| Compound 3 | 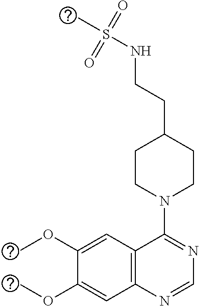 [SMILES: COc1cc2ncnc(N3CCC(CCNS(C)(=O)=O)CC3)c2cc1OC] | 1 | 4.4 | 155 | 35.2 |
| Compound 4 | 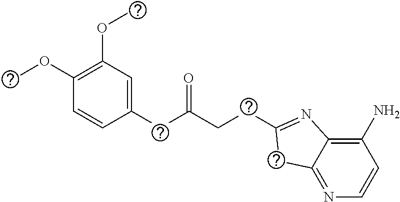 [SMILES: COc1ccc(CC(=O)CCC2=Nc3c(N)ccnc3C2)cc1OC] | 2 | 0.8 | >30000 | >37500 |
| Compound 5 | 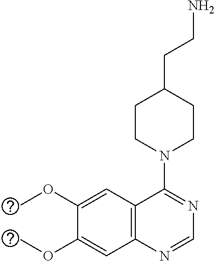 [SMILES: COc1cc2ncnc(N3CCC(CCN)CC3)c2cc1OC] | 1 | 46.7 | >30000 | >642 |
| Compound 6 | 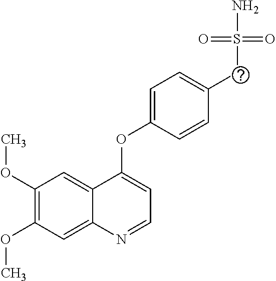 [SMILES: COc1cc2nccc(Oc3ccc(CS(N)(=O)=O)cc3)c2cc1OC] | 1 | 30.7 | >10000 | >325 |
| Compound 7 | 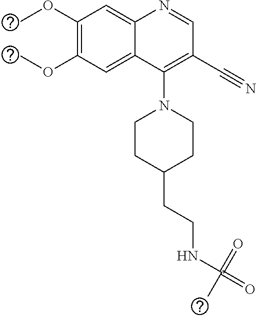 | 1 | 0.7 | 171 | 244 |
| Compound 8 | 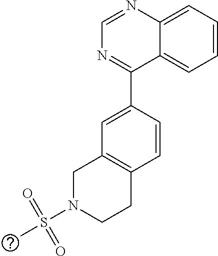 [SMILES: CS(=O)(=O)N1CCc2ccc(-c3ncnc4ccccc34)cc2C1] | 1 | 190 | >30000 | >158 |
| Compound 9 | 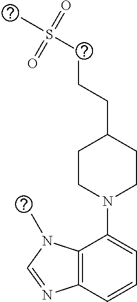 [SMILES: Cn1cnc2cccc(N3CCC(CCCS(C)(=O)=O)CC3)c21] | 1 | 77.7 | >10000 | >128 |
| Compound 10 | 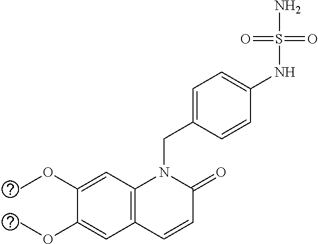 [SMILES: COc1cc2ccc(=O)n(Cc3ccc(NS(N)(=O)=O)cc3)c2cc1OC] | 1 | 234 | >30000 | >127 |
| Compound 11 | 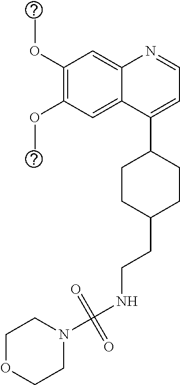 | 1 | 257 | >30000 | >116 |
| Compound 12 | 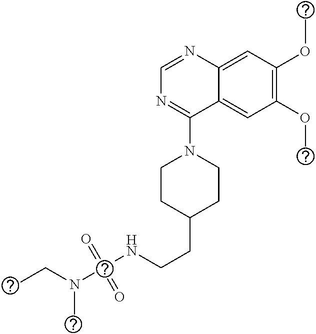 | 1 | 336 | >30000 | >89.0 |
| Compound 13 | 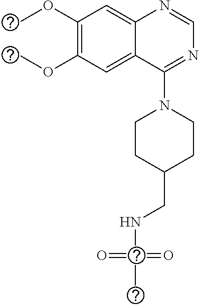 | 1 | 2.4 | 208 | 86.7 |
| Compound 14 | 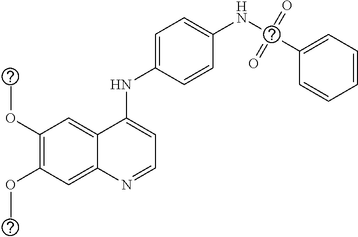 | 1 | 29.1 | 1830 | 62.9 |
| Compound 15 | 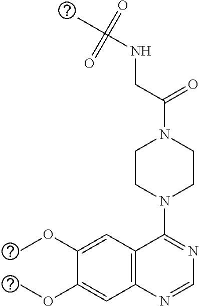 | 1 | 7.3 | 418 | 57.3 |
| Compound 16 | 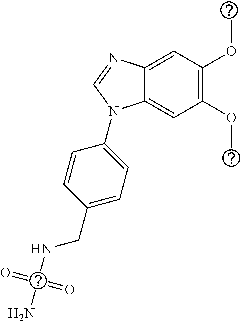 | 1 | 27.1 | 1460 | 53.9 |
| Compound 17 | 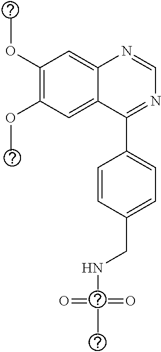 | 1 | 2.9 | 140 | 48.3 |
| Compound 18 | 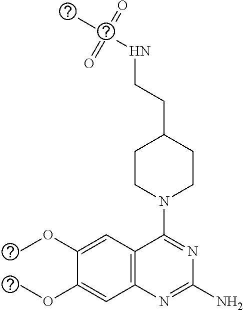 | 1 | 6.2 | 296 | 47.7 |
| Compound 19 | 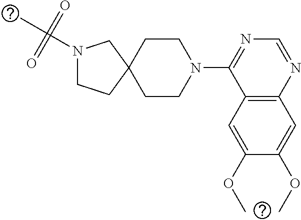 | 1 | 0.8 | 35.8 | 44.8 |
| Compound 20 | 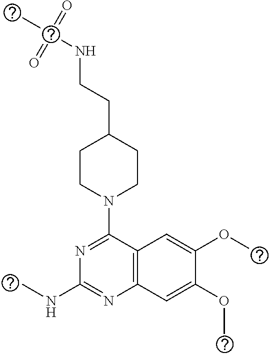 | 1 | 77.5 | >3000 | >38.7 |
| Compound 21 | 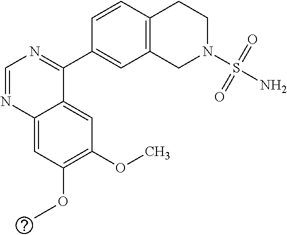 [SMILES: COc1cc2ncnc(-c3ccc4c(c3)CN(S(N)(=O)=O)CC4)c2cc1OC] | 1 | 1.8 | 65.2 | 36.2 |
| Compound 22 | 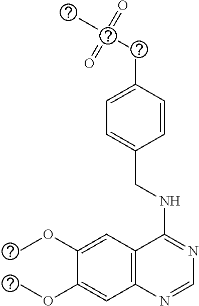 | 1 | 9.1 | 316 | 34.7 |
| Compound 23 | 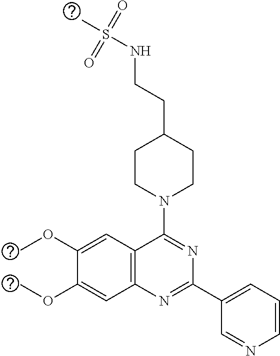 [SMILES: COc1cc2nc(-c3cccnc3)nc(N3CCC(CCNS(C)(=O)=O)CC3)c2cc1OC] | 1 | 418 | >10000 | >23.9 |
| Compound 24 | 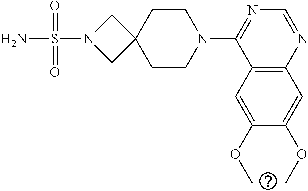 [SMILES: COc1cc2ncnc(N3CCC4(CC3)CN(S(N)(=O)=O)C4)c2cc1OC] | 1 | 60.5 | 1130 | 18.7 |
| Compound 25 | 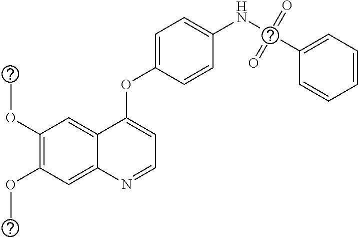 | 1 | 44 | >300 | >6.80 |

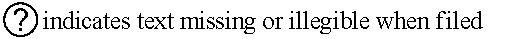

cGAMP Substrate:

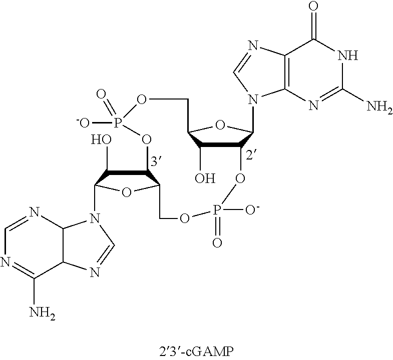

AMP-pNP Substrate:

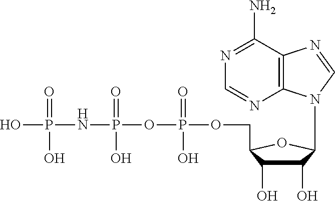

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a subject in need thereof, comprising:
administering to the subject an inhibitor of a 2′3′-cGAMP degradation polypeptide, wherein the inhibitor prevents hydrolysis of 2′3′-cGAMP and wherein the subject has an infection.

2. The method of claim 1, wherein the 2′3′-cGAMP degradation polypeptide is a phosphodiesterase (PDE).

3. The method of claim 2, wherein the PDE comprises an ectonucleotide pyrophosphatase/phosphodiesterase (ENPP) protein.

4. The method of claim 3, wherein the ENPP protein comprises ectonucleotide pyrophosphatase/phosphodiesterase family member 1 (ENPP-1).

5. The method of claim 1, wherein the inhibitor is a small molecule.

6. The method of claim 1, wherein the inhibitor is a PDE inhibitor.

7. The method of claim 1, wherein the inhibitor is a ENPP-1 inhibitor.

8. The method of claim 1, wherein the inhibitor is a reversible inhibitor, a competitive inhibitor, an allosteric inhibitor, a mixed inhibitor, or an irreversible inhibitor.

9. The method of claim 1, wherein the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative, PSB-POM1412-(3H-imidazo[4,5-b]pyridin-2-ylthio)-N-(3,4-dimethoxyphenyl)acetamide or a derivative, analog, or salt thereof; 2-(6-Amino-9H-purin-8-ylthio)-N-(3,4-dimethoxyphenyl)-acetamide, or a salt thereof; N-(3,4-Dimethoxyphenyl)-2-(5-methoxy-3H-imidazo[4,5-b]-pyridin-2-ylthio)acetamide or a salt thereof; 2-(1-(6,7-Dimethoxyquinazolin-4-yl)piperidin-4-yl)ethyl sulfamide or a salt thereof; ((1-(6,7-Dimethoxyquinazolin-4-yl)piperidin-4-yl)methyl)sulfamide or a salt thereof; or SK4A (SAT0037) or a derivative or salt thereof.

10. The method of claim 1, wherein the inhibitor comprises Compound 1, Compound 2, Compound 3, or a derivative, analog, or salt thereof.

11. The method of claim 1, wherein the infection is a viral infection.

12. The method of claim 11, wherein the viral infection is due to a DNA virus, or a retrovirus.

13. The method of claim 11, wherein the viral infection is due to herpes simplex virus 1 (HSV-1), herpes simplex viruses 2 (HSV-2), varicella-zoster virus (VZV), Epstein-Barr virus (EBV), murine gamma-herpesvirus 68 (MHV68), Kaposi's sarcoma-associated herpesvirus (KSHV), human herpesvirus 6A (HHV-6A), human herpesvirus 6B (HHV-6B), human herpesvirus 7 (HHV-7), vaccinia virus (VACV), adenovirus, human papillomaviruses (HPV), hepatitis B virus (HBV), hepatitis D virus (HDV), human immunodeficiency virus (HIV), human cytomegalovirus (HCMV), dengue fever virus, yellow fever virus, ebola virus, Marburg virus, venezuelan equine encephalitis virus, or zika virus.

14. The method of claim 1, wherein the infection is a bacterial infection.

15. The method of claim 14, wherein the bacterial infection is due to a Gram-negative bacterium or a Gram-positive bacterium.

16. The method of claim 14, wherein the bacterial infection is due to Listeria monocytogenes, Mycobacterium tuberculosis, Francisella novicida, Legionella pneumophila, Chlamydia trachomatis, Streptococcus pneumoniae, or Neisseria gonorrhoeae.

17. The method of claim 1, wherein the inhibitor is administered continuously, at predetermined time intervals or intermittently for 1, 2, 3, 4, 5, 6, 7, 8, 9, 10, 14, 15, 28, 30 or more days.

18. The method of claim 1, wherein the inhibitor is administered to the subject at a therapeutically effective amount.

19. The method of claim 18, wherein the therapeutically effective amount of the inhibitor selectively inhibits hydrolysis of 2′3′-cGAMP and has a reduced inhibition function of ATP hydrolysis of the 2′3′-cGAMP degradation polypeptide.

20. The method of claim 1, wherein the subject is a human.

21. A method of enhancing type I interferon (IFN) production in a subject having an infection due to a pathogen, comprising:
administering to the subject having an infection due to a pathogen a pharmaceutical composition comprising:
i) an inhibitor of a 2′3′-cGAMP degradation polypeptide to block the hydrolysis of 2′3′-cGAMP; and
ii) a pharmaceutically acceptable excipient;
wherein the presence of 2′3′-cGAMP activates the STING pathway, thereby enhancing the production of type I interferons.

22. The method of claim 21, wherein the 2′3′-cGAMP degradation polypeptide is a phosphodiesterase (PDE).

23. The method of claim 22, wherein the PDE comprises an ectonucleotide pyrophosphatase/phosphodiesterase (ENPP) protein.

24. The method of claim 23, wherein the ENPP protein comprises ectonucleotide pyrophosphatase/phosphodiesterase family member 1 (ENPP-1).

25. The method of claim 21, wherein the inhibitor is a small molecule.

26. The method of claim 21, wherein the inhibitor is a PDE inhibitor.

27. The method of claim 21, wherein the inhibitor is a ENPP-1 inhibitor.

28. The method of claim 21, wherein the inhibitor is a reversible inhibitor, a competitive inhibitor, an allosteric inhibitor, a mixed inhibitor, or an irreversible inhibitor.

29. The method of claim 21, wherein the inhibitor comprises ARL67156, diadenosine 5′,5″-boranopolyphosphonate, adenosine 5′-(α-borano)-β,γ-methylene triphosphate, adenosine 5′-(γ-thio)-α,β-methylene triphosphate, an oxadiazole derivative, a biscoumarine derivative, reactive blue 2, suramin, a quinazoline-4-piperidine-4-ethylsulfamide derivative, a thioacetamide derivative, PSB-POM1412-(3H-imidazo[4,5-b]pyridin-2-ylthio)-N-(3,4-dimethoxyphenyl)acetamide or a derivative, analog, or salt thereof; 2-(6-Amino-9H-purin-8-ylthio)-N-(3,4-dimethoxyphenyl)-acetamide, or a salt thereof; N-(3,4-Dimethoxyphenyl)-2-(5-methoxy-3H-imidazo[4,5-b]-pyridin-2-ylthio)acetamide or a salt thereof; 2-(1-(6,7-Dimethoxyquinazolin-4-yl)piperidin-4-yl)ethyl sulfamide or a salt thereof; ((1-(6,7-Dimethoxyquinazolin-4-yl)piperidin-4-yl)methyl)sulfamide or a salt thereof; or SK4A (SAT0037) or a derivative or salt thereof.

30. The method of claim 21, wherein the inhibitor comprises Compound 1, Compound 2, Compound 3, or a derivative, analog, or salt thereof.

31. The method of claim 21, wherein the pathogen is a virus.

32. The method of claim 31, wherein the virus is a DNA virus, or a retrovirus.

33. The method of claim 31, wherein the virus is herpes simplex virus 1 (HSV-1), herpes simplex viruses 2 (HSV-2), varicella-zoster virus (VZV), Epstein-Barr virus (EBV), murine gamma-herpesvirus 68 (MHV68), Kaposi's sarcoma-associated herpesvirus (KSHV), human herpesvirus 6A (HHV-6A), human herpesvirus 6B (HHV-6B), human herpesvirus 7 (HHV-7), vaccinia virus (VACV), adenovirus, human papillomaviruses (HPV), hepatitis B virus (HBV), hepatitis D virus (HDV), human immunodeficiency virus (HIV), human cytomegalovirus (HCMV), dengue fever virus, yellow fever virus, ebola virus, Marburg virus, venezuelan equine encephalitis virus, or zika virus.

34. The method of claim 21, wherein the pathogen is a bacterium.

35. The method of claim 34, wherein the bacterium is a Gram-negative bacterium, or a Gram-positive bacterium.

36. The method of claim 34, wherein the bacterium is Listeria monocytogenes, Mycobacterium tuberculosis, Francisella novicida, Legionella pneumophila, Chlamydia trachomatis, Streptococcus pneumoniae, or Neisseria gonorrhoeae.

37. The method of claim 21, wherein the inhibitor is administered continuously, at predetermined time intervals or intermittently for 1, 2, 3, 4, 5, 6, 7, 8, 9, 10, 14, 15, 28, 30 or more days.

38. The method of claim 21, wherein the inhibitor is administered to the subject at a therapeutically effective amount.

39. The method of claim 38, wherein the therapeutically effective amount of the inhibitor selectively inhibits hydrolysis of 2′3′-cGAMP but not ATP hydrolysis in the 2′3′-cGAMP degradation polypeptide.

40. The method of claim 21, wherein the subject is a human.
